(19) United States
(12) Patent Application Publication
(10) Pub. No.: US 2004/0091893 A1
(43) Pub. Date: May 13, 2004

(54) Method for studying the effects of commensal microflora on mammalian intestine and treatments of gastrointestinal-associated disease based thereon

(76) Inventors: Jeffrey Gordon, St. Louis, MI (US); Lora Virginia Hooper, Crestwood, MI (US); Thaddeus Smith Stappenbeck, St. Louis, MI (US); Per Falk, Molndal (SE); Lennart Hansson, Pixbo (SE)

Correspondence Address: NIXON & VANDERHYE, PC, 1100 N GLEBE ROAD, 8TH FLOOR, ARLINGTON, VA, 22201-4714, US

(21) Appl. No.: 10/432,819

(22) PCT Filed: Nov. 27, 2001
(86) PCT No.: PCT/US01/44332

(51) Int. Cl. C12Q 1/68; G01N 33/554; G01N 33/569

(52) U.S. Cl. 435/6; 800/3; 435/7.32

(57) ABSTRACT

a method of investigating chemical changes resulting from commensal microflora colonisation of mammalian intestine which comprises: a) measuring gene expression in commensal bacterium-colonized and germ-free intestine of at least one gene; and b) identifying a gene from a) that has at least a 2-fold difference in expression level between commensal bacterium-colonized and germ-free intestine. The method selects genes for further evaluation, and gives rise to the development of prophylactic treatments.

### TECHNICAL FIELD

The present invention relates to methods for investigating changes resulting from commensal microflora colonisation of mammalian intestine, and the use of information obtained in the production of agents and therapies useful in the modification of the digestive tract and in the treatment of gastrointestinal-associated disease.

### BACKGROUND ART

Mammals generally and humans in particular are home to an incredibly complex and abundant ensemble of microbes. Contact with components of this microflora begin at birth. The human intestine is more densely populated with microbes than any other mucosal surface. Therefore, this organ represents a site where the microflora are likely to have a pronounced influence on host physiology.

Although the effects of pathogenic or other potentially harmful invasive microorganisms have been studied (see for example L. Eckmann et al., J. Biol. Chem. 2000, 275:14084-14094, D. A. Relman, Science, May 21, 1999 : 284 (5418) 1308-10, D. A. Relman, Curr. Opin. Immunol. 2000 April:12 (2):215-8) astonishingly little is known about how commensal bacteria shape normal development and physiology. This is due partly to a paucity of defined, experimentally tractable in vivo model systems for examining how nonpathogenic microorganisms regulate host biology, but also to a prevailing view that these microorganisms had no significant impact on for instance the digestive processes.

A model using adult germ-free animals, colonized with Bacteroides thetaiotaomicron, has previously been used to show that this commensal organism regulates production of ileal epithelial fucosylated glycans after it is introduced into germ-free mice, and to delineate how the microbe controls production of these glycans for its own nutritional benefit (L. Bry, et al., Science 273, 1380, 1996; L. V. Hooper, et al., Proc. Natl. Acad. Sci. USA 96, 9833 (1999). Virtually nothing else is known about how indigenous bacteria modulate intestinal gene expression and how this impact on the host's condition.

The applicants have found that commensal microflora make significant contributions to defining gut physiology and maturation and have developed means for testing this at a molecular level.

### SUMMARY OF THE INVENTION

According to a first aspect of the present invention there is provided a method of investigating changes resulting from commensal microflora colonisation of mammalian intestine which comprises:

a) measuring gene expression in commensal bacterium-colonized and germ-free intestine of at least one gene; and

b) identifying a gene from a) that has at least a 2-fold difference in expression level between intestines colonised by at least one commensal bacterium and germ-free intestines.

Suitably, multiple gene expression is measured by DNA microarray analysis and/or quantitative RT-PCR as illustrated hereinafter, but other conventional methods may be applied. Examples of such methods include quantitative Northern blotting as well as Representational Differentiation Analysis (RDA) which is a PCR based assay which detects genes that differ between two samples (e.g., Odeberg J, et al., Biomol. Eng. 17, 1-9, 2000). In particular, the use of high-density oligonucleotide arrays is preferred for conducting a comprehensive analysis of the range of intestinal functions that are shaped by components of the microflora.

Microarray analysis can be used to measure host responses in a complex tissue composed of multiple cell types, as is found in the intestine. The value of an in vivo model for delineating host cellular responses to a given microbe is that, unlike cell culture-based models, the contributions of lineage and environmental factors to shaping the response are preserved, and may be studied in a germfree experimental system.

In a preferred embodiment, the responding cell population is recovered without perturbing its expressed mRNA population, so that its reaction to the microbe can be characterized in quantitative terms. This is suitably achieved by combining two techniques, laser-capture microdissection (LCM), followed by quantitative analysis for example quantitative PCR and/or microarray analysis of the laser-capture samples.

Using the method of the invention, it is possible to establish the feasibility of assigning an in vivo host response to a particular cell population in a complex tissue, and describing that cellular response in quantitative terms. It may be possible to identify host genes that function as vital but heretofore unappreciated regulators of intestinal biology and host physiology. Genes identified based on their response to colonization by the prototypic gut commensal may represent entirely new targets for therapeutic manipulation. Intestinal microbes have been subjected to great selective pressure over millions of years to find subtle but effective ways of manipulating their host so that both microbe and host benefit from the relationship. Identifying and testing bacterial gene products that affect their host gene targets may yield new pharmacologic agents whose chemical structures and mechanisms of action would, or could not have been appreciated previously.

The germ-free intestine and the commensal bacterium-colonized intestine used in the method of the invention may be in any animal model, but in particular a simplified mouse model of intestinal-microbial interactions is used.

This method allows the effects of individual commensal bacteria to be studied, as well as combinations of these if required.

The applicants have found that genes involved or implicated in the immuno-inflammatory process are not likely to be identified using the method of the invention. This is consistent with the host's need to accommodate resident gut microbes, such as B. thetaiotaomicron, for its entire lifespan. The method of the invention however, can be used to confirm this finding with other commensural bacteria.

Genes identified in this way and their function may then be subject to further study in order to determine their effects, for example on the digestive process. The further investigation may be carried out in for example using any of the conventional methods.

In a preferred embodiment; the method further comprises (c) the step of further investigating a gene identified in b) with regard to its function. Suitable methods by which this can be achieved include work in in vitro cell culture assays or in lower eukaryotic model organisms as well as in animal models such as transgenic animal models.

In vitro cell culture assay techniques will allow studies in a less complex system than the whole animal where the gene and its associated genes and gene products are available for manipulation and analysis.

Lower eukaryotic model organisms such as yeast (e.g., Saccharomyces cerevisae), the fruit fly (Drosophila melanogaster) and the nematode (Caenorhbditis elegans) are eukaryotic organisms with very well defined genomes and in many instances cellular function. Several of the basic signaling functions of interest to study in mammals are conserved also in the simpler eukaryotic life forms which makes them very attractive as models for defined molecular events. These organisms are easier to handle than mammals.

In these systems, the function can be studied using methods such as

i) transgenic knockout;

ii) dominant-negative experiments;

iii) transgene overexpression;

iv) antibody binding assay;

v) by pharmacological intervention using defined chemical agents.

Transgenic knockout methods are based upon inactivation of a gene within an organism, for example using recombinant DNA technology to delete or mutate a gene such that the gene product is dysfunctional, or to introduce an antisense oligonucleotide to silence the gene. In the latter case, antisense oligonucleotides which are complementary to the messenger RNA molecule (mRNA) of the gene are introduced in the organism. The messenger RNA molecule (mRNA) that is the result of transcription of a gene is single stranded and can not be translated into a protein sequence if it is double stranded. Double stranded RNA is formed with the complementary fragment and the resulting double stranded RNA fragment can not be further processed. Thus no protein will be generated from this gene. This is usually done as a transient experiment, i.e., the antisense fragment is added to the cell, model organisms or mammalian model and the gene expression will be silenced as long as there is antisense available. After it is consumed, normal function will be resumed and the normal/control state will be reestablished.

Classical transgenic knockout methods are based on introducing a mutated, and thereby dysfunctional gene into an embryonal cell line. This cell line is then introduced into a normal blastocyst, thereby creating a chimeric fetus consisting of germ cells from the normal background and from the embryonal stem (ES) cell line. Through several rounds of breeding the ES cell-derived mutant allele will be found in the germ cell and thus be transferable to offspring.

New techniques now allow for such mutations to be inherited in a silent and inactive form, so that they can be activated in adult life when development is completed. This is achieved by exposing the adult animal, in particular a mouse, carrying such a silent mutation to a chemical agent (hormone, antibiotic) that will activate expression of the mutated gene.

Another way of achieving a disruption of a gene function is by performing a dominant-negative experiment. In this case a gene that is defective is introduced into the genome of the model organism under transcriptional control of a promotor that will allow very high expression in the targeted cell lineage. The product will by produced and transported to the intended site of action in the cell and compete with the normal gene product for these sites. As the transgene is present in higher amounts and is incapable of performing the intended function, the net outcome will be that this function is perturbed.

The effects of overexpression of the gene in a particular system or model may also be studied. In this case, either multiple gene copies may be introduced into the test organism or use can be made of particular promoters which express genes at high levels.

The classical transgenic experiment involving transgene overexpression includes introducing a gene normally absent from the model (e.g., a human-specific gene into mouse) to assess the effects of the gene on cellular function and/or physiology. If required, the transgene may be placed under the control of tissue-specific promoter sequence so that expression is directed to a cell population of choice. Promoters exist both for a generalised expression throughout the body and for very subtle distribution in specific minor cell population in defined areas of an organ.

Other means of studying the effects of genes involve intervention at the protein level with the gene product. For example, antibody binding assays can be used both to determine whether a protein is present in a cell, model organism or mammal and/or to inhibit the function of the gene product by specifically binding to it and interfering with its capacity to interact with its intended molecular partners.

Pharmacological intervention using defined chemical agents may be particularly useful if the gene can be identified as belonging to a specific class of molecules, e.g., G-protein coupled receptors, proteases or nuclear receptors, its functions can be assessed by using chemicals that are known to act as agonists or antagonists for this type of molecules.

Any commensal bacteria may be used in the method of the invention. Particular examples include Bacteroides thetaiotaomicron, Escherischia coli, Bifidobacterium infantis, or mixtures thereof, or complete ileal and/or cecal microflora obtained from conventionally-raised species. A particularly suitable bacterium for use in the method is B. thetaiotaomicron. The applicants have found that different members of the commensal flora produce different results, indicating a high level of specificity amongst species.

The method of the invention has already revealed an unanticipated breadth of this commensal's impact on gut gene expression. Suitably claim expression of a wide range of genes is measured in step (a) of the method of the invention. Examples of such genes include genes associated with the nutrient uptake and metabolism, hormone/maturational responses, mucosal barrier function, detoxification/drug resistance, xenobiotic metabolism, motility, enteric nervous system/muscular layer development or activity, angiogenesis, cytoskeleton/extracellular matrix function or development, signal transduction and other essential cellular functions.

Nutrient uptake and metabolism genes which may be the subject of study include genes associated with carbohydrate uptake and metabolism such as Na+/glucose cotransporter (SGLT1) or lactase phlorizin-hydrolase genes, genes associated with lipid uptake and metabolism such as pancreatic lipase-related protein 2, colipase, liver fatty acid binding protein, fasting induced adipose factor (FIAF), apolipoprotein A-IV, phospholipase B and CYP27 genes, metal uptake or sequestration genes such as high-affinity copper transporter, metallothionein I, metallothionein II or ferritin heavy chain genes, or cellular energy production such as isocitrate dehydrogenase subunit, succinyl CoA transferase, transketolase, malate oxidoreductase and aspartate aminotransferase genes.

Examples of genes associated with hormonal/maturational responses include adenosine deaminase, omithine decarboxylase antizyme, 15-hydroxyprostaglandin dehydrogenase, GARG-16, FKBP51, androgen-regulated vas deferens protein, short chain dehydrogenase and heat-stable antigen genes.

Examples of genes associated with mucosal barrier function include decay-accelerating factor, polymeric Ig receptor, small proline-rich protein 2a, serum amyloid A protein, CRP-ductinα (MUCLIN), zeta proteasome chain, and anti-DNA IgG light chain genes.

Suitable genes which are involved in detoxification/drug resistance include glutathione S-transferase, P-glycoprotein (mdr1a) and CYP2D2 genes.

Examples of genes associated with enteric nervous system/muscular layer development or activity include L-glutamate transporter, L-glutamate decarboxylase, vesicle-associated protein-33, cysteine-rich protein 2, smooth muscle (enteric) gamma actin and SM-20 genes.

RNAse super family members include several angiogenins. They also include the angiogenin-4, which the applicants have sequenced and found to be particularly interesting in that it is intestine-specific, epithelial-based and its expression appears to be regulated by components of the microbiota. The sequence of murine angiogenin-4 has recently been published (D. E. Holloway et al., Protein Expression and Purification, (2001), 22:307.

The applicants have surprisingly found that one such gene, specifically the angiogenin-4 gene is expressed in intestinal epithelium and that expression levels are particularly affected by commensal bacteria.

Examples of genes associated with cytoskeleton/extracellular matrix function include gelsolin, destrin, alpha cardiac actin, endoB cytokeratin, fibronectin, proteinase inhibitor 6 and alpha 1 type 1 collagen genes.

Signal transduction genes include Pten, gp106 (TB2/DP1), rac2, Semcap2, serum and glucocorticoid-regulated kinase, STE20-like protein kinase and B-cell myeloid kinase. (The signal pathway in which rac2 gene is associated in known to have an impact on the mucosal barrier function).

Finally, other examples of genes associated with essential cellular functions include glutathione reductase, calmodulin, elF3 subunit, hsc70, oligosaccharyl transferase subunit, fibrillarin, H+-transporting ATPase and Msec23 genes.

Suitably in the method of the invention, at least 10 such genes are measured in step (a). Preferably, expression of all the genes listed above are measured.

In accordance with the invention, genes with at least a 2-fold difference in expression are identified and selected for further study. Suitably, genes for which at least a 4-fold difference, more suitably at least a 5-fold difference, yet more suitably a 7-fold difference and preferably a 9-fold difference in expression are identified in step (b) and are selected for further study.

Some particular genes which have already been identified using the method of the invention following colonisation with B. thetaiotaomicron and are of particular interest. These include colipase, decay-accelerating factor (DAF), the polymeric IgA receptor, small proline-rich protein 2a (Sprr2a), {angiogenin-3,} Pten, CYP2D2, Sprr2a, rac2, and Mdr-1. They also include angiogenin-4, a newly discovered protein which is related to angiogenin-3.

These genes may have useful therapeutic functions and thus, the expression of one or more of these genes is preferably measured in the method of the invention in order to detect the impact of the particular commensal bacteria undergoing study on their expression.

In particular, the method of the invention has found that expression of colipase and angiogenins such as the angiogenin whose gene is amplifiable using primers such as SEQ ID NO 12 and 25 (see Table 3 hereinafter) which is angiogenin-4, as well as Sprr2a and rac2 should be subject to further investigation.

Thus, the invention further comprises evaluation of the biochemical pathway in which the angiogenin whose gene is amplifiable using primers such as SEQ ID NO 12 and 25 (see Table 3 hereinafter) which is angiogenin-4, participates in the intestine.

In an alternative embodiment, the invention further comprises evaluation of the biochemical pathway in which colipase participates in the intestine.

In an alternative embodiment, the invention further comprises evaluation of the biochemical pathway in which Sprr2a participates in the intestine.

In an alternative embodiment, the invention further comprises evaluation of the biochemical pathway in which rac2 participates in the intestine.

Evaluation can be carried out using any suitable method, including those described above.

Identification of genes using the method of the invention as well as further study of the biochemical pathways associated with these, could lead to prophylactic or therapeutic treatment of disease or disorders of the gastrointestinal tract. In particular, by identifying which genes are most affected by the presence or absence of commensal bacteria and which are involved in a biochemical pathway associated with a condition, disease or disorder, it would be possible to devise treatments aimed at altering expression of a particular gene in order to rectify that condition, disease or disorder. Alternatively, it would be possible to intervene pharmacologically in the pathways maintained by the gene products.

This may be effected by administration of an appropriate commensal bacteria. Increased populations of desirable microflora may be achieved by administration of the bacteria in oral form, such as in the form of tablets, pharmaceutical or nutriceutical compositions or even foodstuffs such as live yoghurt cultures

In particular, the invention provides a method of modulating epithelialy-expressed angiogenesis factor by colonisation with a commensal bacteria which effects said modulation.

Another particular example of such a methods is a method of modifying metabolism, in particular of dietary lipids, which method involves use of a commensal bacteria identified using a method as described above, as having an effect on said metabolism.

Yet another particular example is a method of modifying epithelial barrier function using a commensal bacteria identified using the above-described method.

Another example is a method of preventing or treating tumors of the intestine, by modifying the population of commensal bacteria present therein which bacteria have been identified using the method of the invention as modulating angiogenesis, or signal transduction.

In a further aspect, the method of the invention may be useful in diagnosis of disease or conditions caused by inappropriate levels of gene expression in the gut. Analysis of commensal microflora taken from a patient will show a high degree of natural variation in the populations of microflora as discussed above. However, the detection of particularly elevated or reduced levels of commensal microflora identified using the method of the invention may indicate that a particular gene is being expressed at abnormal levels, giving rise to a disease state or condition. Treatment of such conditions may be effected either by altering the levels of the commensal bacteria as appropriate and as discussed above, or by direct administration of a bacterial or human gene product or derivative, or of means to block the gene product at the protein level, such as using chemical or biological inhibitors or antagonists of the gene product.

The method of the invention can be used widely to address a question that applies to humans and innumerable other species that reside in our microbe-dominated world, namely how do bacteria contribute to and regulate the physiology and maturation of their hosts? In the case of humans, assembly and maintenance of a microflora undoubtedly involves intricate combinatorial regulatory mechanisms, developed over the course of a long selective process that involved co-evolution of our predecessors with their microbial partners. The results presented below demonstrate the impact of an indigenous bacterial species on expression of genes that participate in vital physiologic functions, and emphasize the importance of viewing our biology as intertwined with the biology of our complex assemblies of resident bacteria.

The results also demonstrate the practicality of using defined in vivo models to deduce the responses of specified cellular populations within complex tissues to microbes, in a manner that preserves the influence of the surrounding cellular and environmental milieu.

These models and approaches will allow the pervasive contribution of microbes to human health to be evaluated and microbial products that are useful therapeutically to be identified. In addition, they should reveal how these normal host-microbial relationships affect various disease processes, and provide new perspectives and definitions about what constitutes a pathogenic relationship.

In addition however, the method has already revealed that a number of genes or gene products appear to have a significant effects in intestinal tissue, giving rise to the possibility that pharmaceuticals could be developed to target such genes or gene products in a manner which is beneficial to a patient. This can be done by screening for compounds which modulate the activity of the gene product.

A further aspect of the invention comprises a method for identifying genes that function as regulators of intestinal biology, said method comprising applying the method as described above and detecting expression genes which have not heretofore been associated with such function.

Thus a further aspect of the invention comprises a method of screening compounds having a pharmaceutical application in a gastrointestinal disease, which method comprises assaying the compounds for their ability to modulate the activity of the product of a gene identified using a method described above.

Yet a further aspect of the invention is a method of treating or preventing gastrointestinal disease which method comprises administering a therapeutically effective amount of a compound which modulates the activity of the product of a gene identified using a method according to claim 1.

In particular, on the basis of the results reported hereinafter, the invention further provides a method of screening for a compound potentially useful for treatment or prophylaxis of conditions characterized by a defect in intestinal barrier function which comprises assay of the compound for its ability to modulate the activity or amount of small proline-rich protein 2a (sprr2a) or rac2.

Suitable screening methods would be apparent to the skilled person. Once identified, the compounds are useful in the treatment or prophylaxis of conditions in which intestinal barrier function is comprised.

Thus a further aspect of the invention comprises the use of a compound able to modulate the activity or amount of small proline-rich protein 2a (sprr2a) or rac2 in preparation of a medicament for the treatment or prophylaxis of conditions characterized by a defect in intestinal barrier function.

Suitably, the compounds will be formulated as pharmaceutical compositions. Novel compositions of this type and their preparation form a further aspect of the invention.

### BRIEF DESCRIPTION OF THE DRAWINGS

FIG. 1 shows the results of real-time quantitative RT-PCR studies of colonization-associated changes in gene expression in laser capture microdissected ileal cell populations, LCM of the ileum of a colonized mouse. Sections were stained with nuclear fast red. Bars=25 μm.

FIG. 2 shows the results of real-time quantitative RT-PCR analyses of mRNA levels in isolated from laser-captured cell populations. Values are expressed relative to levels in germ-free mesenchyme using ΔΔC_{T} analysis described below. Each gene product per sample was assayed in triplicate in 3-4 independent experiments. Representative results (mean±1 S.D.) from pairs of germ-free and colonized mice are plotted.

FIG. 3 shows the results of an experiment to illustrate the specificity of host responses to colonization with different members of the microflora. Germ-free mice were inoculated with one of the indicated organisms, or with a complete ileal/cecal flora from conventionally raised mice (Conv. microflora) (4). Ileal RNAs, prepared from animals colonized at =10⁷ CFU/ml ileal contents 10 days after inoculation, were pooled, and levels of each mRNA shown were analyzed by real time quantitative RT-PCR (qRT-PCR). Mean values (±1S.D.) for triplicate determinations are plotted.

FIG. 4 shows the nucleotide sequences of mouse angiogenin-4 and angiogenin-3 in alignment (SEQ ID NOS 29 and 30 respectively).

FIG. 5 illustrates the sequence alignment of the amino acid sequences of mouse angiogenin family members (SEQ ID NOS 31-34).

FIG. 6 shows the locations of primers specific for mouse angiogenin family members.

FIG. 7 is a graph illustrating tissue distribution of angiogenin-4 mRNA,. together with the results of an agarose gel analysis.

FIG. 8 is a graph illustrating tissue distribution of angiogenin-1 mRNA.

FIG. 9 is a graph illustrating tissue distribution of angiogenin-3 mRNA following quantitative real-time RT-PCR analysis.

FIG. 10 shows the results of RT-PCR analysis showing the absence of angiogenin-related protein expression.

FIG. 11 is a set of graphs showing the results of experiments on the microbial regulation of angiogenin-4 expression in the small intestine.

FIG. 12 is a graph showing the regulation of angiogenin-4 expression during postnatal development.

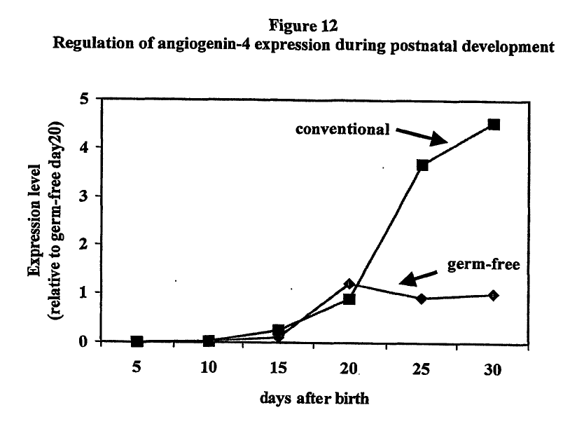

FIG. 13 is a block graph showing cellular localization of angiogenin-4 expression in small intestine: qRT-PCR analysis of cells isolated from the crypt base.

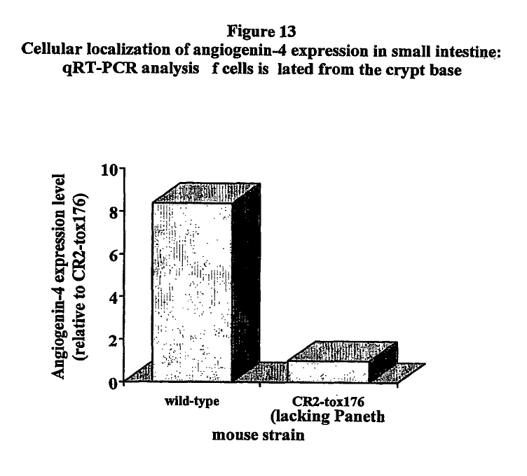

### DETAILED DESCRIPTION OF THE INVENTION

In order to study at a molecular level, the changes in the intestine orchestrated by commensal bacteria, germ-free mice were colonised with commensal bacteria including Bacteroides thetaiotaomicron, a prominent component of the normal mouse and human intestinal microflora.

Global intestinal transcriptional responses to colonization were delineated using high-density oligonucleotide arrays and the cellular origins of specific responses established by laser capture microdissection and real-time quantitative RT-PCR.

The results illustrated hereinafter, reveal that commensal bacteria modulate expression of a large number of genes, some to significant levels. The genes involved participate in diverse and fundamental physiological functions of the gut, including nutrient absorption, mucosal barrier fortification, and xenobiotic metabolism. The species-selectivity of some of the colonization-associated changes in gene expression emphasizes how human physiology can be impacted by changes in the composition of indigenous microflora.

Furthermore, it would appear that some commensals play a role in postnatal developmental transitions. Changes associated with the suckling-weaning transition were elicited in adult mice by B. thetaiotaomicron, suggesting that indigenous bacteria play an instructive role in postnatal gut development.

Coupling defined in vivo models with comprehensive genome-based analyses thus provides a powerful approach for identifying the critical contributions of resident microbes to host biology.

Bacteroides thetaiotaomicron is a genetically-manipulatable anaerobe and was chosen for initial study to define the impact of resident bacteria on intestinal biology because it is a prominent member of both the adult mouse and human gut microflora. Moreover, B. thetaiotaomicron normally colonizes the distal small intestine (ileum) during the suckling-weaning transition, a time of rapid and pronounced functional maturation of the gut (W. E. C. Moore, L. V. Holdeman, Appl. Microbiol. 27, 961 (1974), T. Ushijima, M. Takahashi, K. Tatewaki, Y. Ozaki, Microbiol. Immunol. 27, 985 (1983)).

Colonization elicited a concerted response involving enhanced expression of four components of the host's lipid absorption machinery. mRNAs encoding pancreatic lipase related protein-2 (PLRP-2) and colipase increased an average of 4- and 9-fold, respectively (Tables 1 and 2). PLRP-2 hydrolyzes tri- and diacylglycerols, phospholipids and galactolipids. Colipase augments PLRP-2 activity (M. E. Lowe, M. H. Kaplan, L. Jackson-Grusby, D. D'Agostino, M. J. Grusby, J. Biol. Chem. 273, 31215 (1998)). In addition, there was a 4-6-fold increase in L-FABP mRNA, which encodes an abundant cytosolic protein involved in fatty acid trafficking within enterocytes, and an induction of apolipoprotein AIV, a prominent component of triglyceride-rich lipoproteins (chylomicrons, VLDL) secreted from the basolateral surfaces of enterocytes (Table 1).

Colonization led to an increase in ileal levels of Na⁺/glucose cotransporter (SGLT-1) mRNA. There was also a concerted rise in expression of several components of the host's lipid absorption/export machinery, including pancreatic lipase-related protein-2 (PLRP-2), colipase, liver fatty acid binding protein (L-FABP), and apolipoprotein A-IV (See Table 1 hereinafter). The prominent decrease in expression of fasting-induced adipose factor, a novel PPARα target known to be repressed with fat feeding (S. Kersten, et al., J. Biol. Chem. 275, 28488 (2000).), provided further evidence for augmented lipid uptake in colonized mice.

The changes in expression of these 6 genes in particular indicate that B. thetaiotaomicron elicits an increased host capacity for nutrient absorption/processing and may provide a partial explanation as to why germ-free rodents require a higher caloric intake to maintain their weight than those with a normal microflora (B. S. Wostmann, C. Larkin, A. Moriarty, E. Bruckner-Kardoss, Lab. Anim. Sci. 33, 46 (1983).

Additionally, the applicants have found that colonisation produces changes in expression of four genes involved in dietary metal absorption. A high affinity epithelial copper transporter (CRT1) mRNA was increased, while metallothionein-I, metallothionein-II, and ferritin heavy chain mRNAs were decreased (Table 1). These changes suggest that colonization engenders increased capacity to absorb heavy metals (e.g., via CRT1) and a concomitant decreased capacity to sequester them within cells (MT-I/II, ferritin). This implies greater host demand for these compounds, either due to increased utilization by the host's own metabolic pathways or to competition with the microbe. The changes in SGLT-1, colipase, L-FABP, and MT1 (plus 8 other mRNAs discussed below), were independently validated by qRT-PCR (C. A. Heid, J. Stevens, K. J. Livak, P. M. Williams, Genome Res. 6, 986 (1996) (see Table 2 below).

Colipase plays a critical role in dietary lipid metabolism by stimulating the activity of both pancreatic triglyceride lipase and PLRP-2. Furthermore, proteolytic cleavage of procolipase yields a pentapeptide (enterostatin) that functions as a satiety signal for fat ingestion (S. Okada, D. A. York, G. A. Bray, Physiol. Behav. 49, 1185 (1991)). The significantly elevated expression found following colonisation with B. thetoaiotaomicron illustrated hereinafter are indicative of a previously unappreciated mechanism by which the intestinal epithelium, together with a resident gut bacterium, contributes to dietary lipid metabolism.

An intact mucosal barrier is critical for accomodating the vast population of resident intestinal microbes. Its disruption can provoke an immune response that is deleterious to the host and to the stability of luminal microflora, leading to pathologic states such as inflammatory bowel disease (P. G. Falk, et al., Microbiol. Mol. Biol. Rev. 62, 1157 (1998)).

It has been found that colonization with B. thetaiotaomicron produces no detectable inflammatory response, as judged by histologic surveys (L. Bry, P. G. Falk, T. Midtvedt, J. I. Gordon, Science 273, 1380 (1996). Moreover, there is no discernible induction (or repression) of the many genes, represented on the microarrays, that are involved in responses. An influx of IgA-producing B-cells does occur in the ileal mucosa 10 days after introduction of B. thetaiotaomicron; similar commensal-induced IgA responses have been shown to be T-cell independent and to enforce barrier integrity (A. J. Macpherson et al., Science 288, 2222 (2000).

Genes involved in barrier function account for 10% (7/71) of the changes in gene expression observed with B. thetaiotaomicron colonization. Microarray and qRT-PCR analyses revealed that the influx of IgA producing B-cells is accompanied by increased expression of the polymeric immunoglobulin receptor (pIgR) that transports IgA across the epithelium (Tables 1,2). There is also augmented expression of the CRP-ductin gene, encoding both a component of the protective mucus layer overlying the epithelium (MUCLIN; R. C. DeLisle, et al., Am. J. Physiol. 275, G219 (1998)) and a putative receptor for trefoil peptides that participate in fortification/healing of the intestinal mucosa (L. Thim, E. MØrtz, Regul. Pept. 90, 61 (2000). Additionally, there is increased expression of decay accelerating factor (DAF), an apical epithelial surface protein that inhibits complement-mediated cytolysis (M. E. Medof, et al, J. Exp. Med. 165, 848 (1987). Coincident enhancement of pIgR, MUCLIN, and DAF expression should not only help prevent bacteria from crossing the epithelial barrier, but should also prevent mucosal damage that may ensue from microbial activation of complement components present in intestinal secretions.

It has been found that decay accelerating factor (DAF) expression increased 5-fold with colonization using B. thetaiotaomicron. DAF is known to be present on the apical surface of intestinal epithelial cells and to inhibit complement-mediated cytolysis (M. E. Medof, E. I. Walter, J. L. Rutgers, D. M. Knowles, V. Nussenzeig, J. Exp. Med. 165, 848 (1987),). The coincident enhancement of DAF, pIgR, and MUCLIN expression should not only help prevent bacteria from crossing the epithelial barrier, but also prevent any mucosal damage that may ensue from microbial activation of complement components present in intestinal secretions.

The most pronounced response to B. thetaiotaomicron was an increase in small proline-rich protein-2 (sprr2a) mRNA (Table 1). qRT-PCR analysis established that there is a 205±64-fold elevation in this mRNA with colonization (Table 2). Sprr2a is a member of a family of proteins associated with terminal differentiation of squamous epithelial cells. Sprrs contribute to the barrier functions of squamous epithelia, both as a component of the cornified cell envelope, and as cross-bridging proteins linked to desmosomal desmoplakin , a prominent desmosomal constituent (P. M. Steinert, L. N. Marekov, Mol. Biol. Cell 10, 4247 (1999). Colonization did not produce a notable change (i.e. two fold or more), in the expression of genes encoding other proteins linked to desmosomes (desmoplakin, plakoglobin, plakophilin, plectin), or tight junctions (ZO-1, occludin).

Sprr2a expression in the intestine and its microbial regulation are novel findings. The critical contribution of sprr2a to the squamous epithelial barrier and the dramatic response of sprr2a expression to B. thetaiotaomicron together suggest that this protein plays an important role in intestinal barrier function. It is therefore a particularly suitable target for further investigation in accordance with the invention, in particular by evaluating the biochemical pathway in which Sprr2a participates in the intestine.

A prominent marker of the weaning transition is the decline in lactase-phlorizin hydrolase (LPH), an enterocytic brush-border enzyme that hydrolyzes the principal milk sugar, lactose. LPH mRNA levels rise throughout the small intestine of conventionally raised animals during the suckling period, and then fall in the ileum during weaning (S. D. Krasinski et al., Am. J. Physiol. 267, G584 (1994)). The effects of commensal bacteria on expression of these genes in particular may be of interest in determining whether the bacteria have signficant impact.

Using the method of the invention, it has been found that colonization results in increased expression of angiogenin-4 which resembles angiogenin-3, a secreted protein with demonstrated angiogenic activity (X. Fu, et al., Mol. Cell Biol. 17, 1503 (1997), X. Fu, et al., Growth Factors 17, 125 (1999)). The 11-fold increase in expression of the angiogenesis factor recognizable by amplification using primers of SEQ ID NO 12 and SEQ ID NO 25, which is angiogenin-4 (Table 1,2) upon B. thetaiotaomicron colonization represents a novel mode of regulation for an angiogenesis factor and so may be the subject of further investigation in accordance with the invention.

Laser capture microdissection (LCM) experiments described below have delineated the cellular origins of this response. This suggests that the presence of bacteria influences intestinal vascularization.

The gut is the site of first contact of innumerable ingested toxins and xenobiotics. The relative contributions of luminal bacteria and the epithelium to detoxification and metabolism of these compounds has been difficult to delineate in conventionally-raised mammals. It has been found that colonization of germ-free mice with B. thetaiotaomicron results in reduced expression of several genes involved in these processes (Table 1 below). There is a decrease in the mRNA encoding glutathione S-transferase, which detoxifies a variety of electrophiles, and a corresponding decrease in multi-drug resistance protein-1 (Mdr-1), which exports glutathione-conjugated compounds from the epithelium (R. W. Johnstone, A. A. Ruefli, M. J. Smyth, Trends Biochem. Sci. 25, 1 (2000)). Expression of CYP2D2 (debrisoquine hydroxylase)involved in oxidative drug metabolism in humans (M. Ingelman-Sundberg, et al., Trends Pharmacol. Sci. 20, 342 (1999), also declines with colonization. Reduced expression of these genes indicates that B. thetaiotaomicron may inself contribute to detoxification of compounds that are deleterious to the host.

A genetic polymorphism that produces a deficiency in this cytochrome P-450 is common in humans and associated with altered oxidative drug metabolism (M. Ingelman-Sundberg, M. Oscarson, R. A. McLellan, Trends Pharmacol. Sci. 20, 342 (1999)). The reduced expression of these three host genes suggests that commensal bacteria such as B. thetaiotaomicron contributes to the detoxification of compounds that could be deleterious to the host. This indicates that a component of the normal microflora can modulate host genes involved in drug metabolism, and underscore how variations in such metabolism between individuals may arise from differences in their resident gut flora. Consequently, evaluation of the effect on commensal bacteria on expression of these genes using the method of the invention may be helpful

Pten is a member of a family of dual specificity protein phosphatases. PTEN haploinsufficiency in humans is associated with increased susceptibility to tumorigenesis (D. J. Marsh et al., Hum. Mol. Genet. 7, 507 (1998)). Furthermore, Pten+/−⇄Pten+/+chimeric mice develop colonic polyps and adenocarcinoma (A. DiCristofano, B. Pesce, C. Cordon-Cardo, P. P. Pandolfi, Nat. Genet. 19, 348 (1998)). The human homolog of Gp106, TB2/DP1, is a component of a locus which when mutated produces multiple intestinal adenomas (R. W. Burt, Adv. Exp. Med. Biol. 470, 99 (1999)). The finding that a component of the microflora affects expression of genes such as the angiogenesis factor whose gene is amplifiable using primers such as SEQ ID NO 12 and 25 (see Table 3 hereinafter) which is angiogenin-4, Pten and Gp106 highlights the importance of considering mechanisms by which intestinal bacteria may contribute to the initiation or progression of tumorigenesis within, or even outside, the gut.

The motility of the intestine is regulated by its enteric nervous system (ENS). The relative contributions of intrinsic and extrinsic factors to ENS activity are poorly understood, despite the fact that irritable bowel syndrome, which involves dysregulated motor activity, is a major health problem. The impact of commensal bacteria such as B. thetaiotaomicron on gut physiology extends to genes expressed in the enteric nervous system (ENS) and in the muscular layers. mRNAs encoding the L-glutamate transporter and L-glutamate decarboxylase, which converts glutamate to GABA, are both increased, suggesting a colonization-associated effect on the glutamatergic neurons of the ENS (M. T. Liu, J. D. Rothstein, M. D. Gershon, A. L. Kirchgessner, J. Neurosci. 17, 4764 (1997)). Enhanced expression of vesicle-associated protein-33, a synaptobrevin-binding protein involved in neurotransmitter release (P. A. Skehel et al., Proc. Natl. Acad. Sci. U.S.A. 97, 1101 (2000) is also observed. There is a concomitant increase in two muscle-specific mRNAs: enteric γ-actin and cysteine-rich protein 2. Previous electrophysiological studies of germ-free and conventionally-raised animals have suggested that the microflora plays a role in gut motility (E. Husebye, P. M. Hellstrom, T. Midtvedt, Dig. Dis. Sci. 39, 946 (1994)). The method of the invention can provide molecular details about how resident gut microbes, such as B. thetaiotaomicron, may act to modulate motility.

B. thetaiotaomicron normally colonizes mouse and human intestine during weaning (W. E. C. Moore, L. V. Holdeman, Appl. Microbial. 27, 961 (1974), T. Ushijima, M. Takahashi, K. Tatewaki, Y. Ozaki, Microbiol. Immunol. 27, 985 (1983)). This period is characterized by a dramatic shift in the composition of the microflora and by a series of critical developmental changes in the intestinal epithelium. It is unclear how many of these changes are regulated by intrinsic cellular mechanisms, and how many are controlled by extrinsic signals emanating from the mesenchyme, or from luminal (dietary, microbial) factors.

Expression profiling revealed surprisingly that colonization of adult germ-free mice with B. thetaiotaomicron elicits other responses that mimic changes which normally occur in the maturing intestine of conventionally-reared animals. Expression of lactase, which hydrolyzes the principal milk sugar (lactose), normally declines during the weaning period (S. D. Krasinski et al., Am. J. Physiol. 267, G584 (1994). Colonization of adult germ-free mice with B. thetaiotaomicron produces a decrease in ileal lactase mRNA (Table 1,2 below). These findings indicate how members of the emerging postnatal normal flora may contribute to intestinal maturation.

Adenosine deaminase (ADA) and polyamines (spermine, spermidine) play important roles in postnatal intestinal maturation (G. D. Luk, L. J. Marton, S. B. Baylin, Science 210, 195 (1980), J. M. Chinsky, et al., Differentiation 42, 172 (1990)). It has been found that B. thetaiotaomicron colonization produces an increase in mRNAs encoding ADA and ornithine decarboxylase (ODC) antizyme but not a 5-fold increase. The antizyme, whose expression is affected by polyamine levels, is a critical regulator of ODC turnover (J. Nilsson, S. Koskiniemi, K. Persson, B. Grahn, I. Holm, Eur. J. Biochem. 250, 223 (1997)); an increase in antizyme mRNA levels therefore suggests that colonization influences ileal polyamine synthesis. These data demonstrate that genes controlling synthesis of two classes of regulators of gut maturation, adenosine and polyamines, are themselves modulated by a component of the microflora, leading to the idea that bacteria serve as upstream effectors of a cascade that affects gut maturation. Other colonization experiments, described below, indicate that other gut commensals have the capacity to engineer such changes.

Changes in gut maturation associated with the suckling-weaning transition are also thought to be regulated by increases in glucocorticoids (S. J. Henning, D. C. Rubin, R. J. Shulman, in Physiology of the Gastrointestinal Tract, L. R. Johnson, Ed. (Raven Press, New York, 1994), pp. 584-586). B. thetaiotaomicron colonization as described hereinafter was accompanied by reduced expression of two genes whose transcription is known to be suppressed by glucocorticoids: 15-hydroxyprostaglandin dehydrogenase (M. D. Mitchell, V. Goodwin, S. Mesnage, J. A. Keelan, Prostaglandins Leukot. Essent. Fatty Acids 62, 1 (2000)) and glucocorticoid-attenuated response gene-16 (J. B. Smith, H. R. Herschman, J. Biol. Chem 270, 16756 (1995)). Furthermore, there was reduced expression of another gene whose product interacts with nuclear hormone receptor family members, the immunophilin FKBP51 (S. C. Nair et al., Mol. Cell. Biol. 17, 594 (1997)). However, the reduction was not greater than 5-fold in any individual case. Thus, other commensal bacteria may be investigated using the method of the invention to see if these could produce a more significant effect.

As mentioned above, the applicants have found that a particular member of the angiogenin family, whose gene is amplifiable using primers of SEQ ID NO 12 and 25 above and is expressed in mouse intestine, is novel. Thus this protein and the gene encoding it forms a further aspect of the invention.

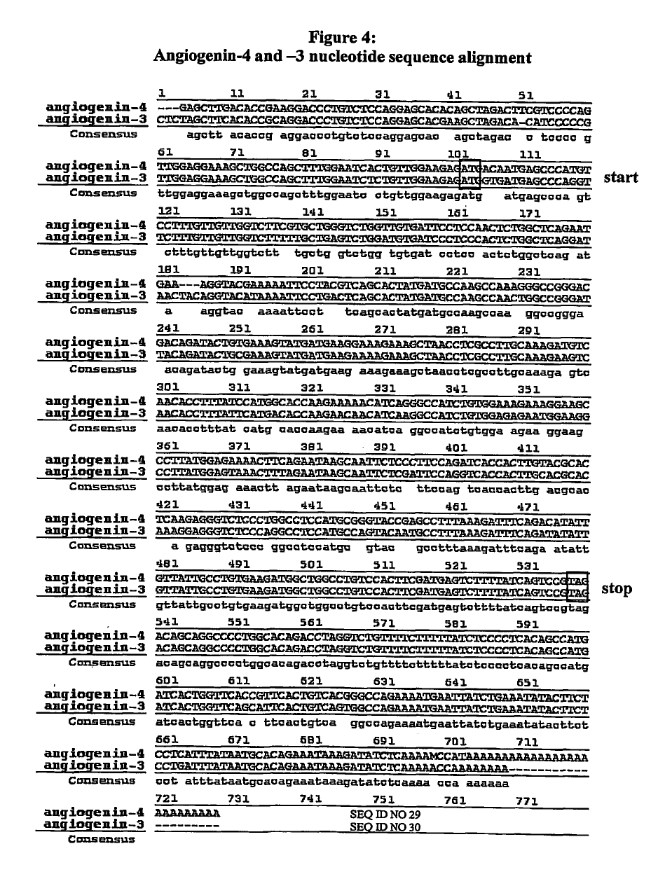

A further aspect of the invention provides a protein of SEQ ID NO 29 as shown in FIG. 4 hereinafter, or an allelic variant thereof or a protein which has at least 85% amino acid sequence identity with SEQ ID NO 29.

In particular, the invention provides a protein of SEQ ID NO 29.

In yet a further aspect, the invention provides a nucleic acid which encodes a protein as described above.

These proteins are useful as a target for the screening process of the invention.

The following Examples illustrate the invention.

### EXAMPLE 1

Age-matched groups of 7-15 week-old germ-free NMRI/KI mice were maintained in plastic gnotobiotic isolators on a 12 hour light cycle, and given free access to an autoclaved chow diet (B&K Universal). Males were inoculated with wild-type B. thetaiotaomicron (strain VPI-5482) (L. Hooper et al (1999) supra). Mice were sacrificed 10 days later, 2 hours after lights were turned on. The distal 1 cm of the small intestine was used to define the number of colony forming units per ml of extruded luminal contents.

Ileal RNA was isolated from mice with >10⁷ colony forming units (CFU) of bacteria per ml of luminal contents. [Earlier studies had shown that 10 days was sufficient to produce robust colonization of the ileum and that =10⁷ CFU/ml were necessary for full induction of fucosylated glycan production in the ileal epithelium (L Hooper et al, (1999) supra. L. Bry, P. G. Falk, T. Midtvedt, J. I. Gordon, Science 273, 1380 (1996))].

Total ileal RNA samples, prepared from the 3 cm of intestine adjacent the distal 1 cm of the small intestine of 4 mice from 3 independent colonizations, and from age- and gender-matched germ-free mice (n=8), using a RNA (Qiagen RNeasy kit),were each pooled, in equal amounts, for generation of biotinylated cRNA targets. Two targets were prepared, independently, from 30 μg of each total cellular RNA pool, using the method outlined by C. K. Lee, et al., Science 285, 1390 (1999).

SYBR green-based real-time quantitative RT-PCR studies ( N. Steuerwaldet al., Mol. Hum. Reprod. 5, 1034 (1999)) were performed using the gene-specific primers listed in Table 3 below and DNAse-treated RNAs.

Control experiments established that the signal for each amplicon was derived from cDNA and not from primer dimers or genomic DNA. Signals were normalized to an internal reference mRNA (glyceraldehyde 3-phosphate dehydrogenase). The normalized data were used to quantitate the levels of a given mRNA in germ-free and colonized ileums (ΔΔC_{T} analysis; Bulletin #2, ABI Prism 7700 Sequence Detection System).

TABLE 3

| | | | | |
|---|---|---|---|---|
| | | SEQ | | SEQ |
| | | ID | | ID |
| gene name | forward primer | NO | reverse primer | NO |

| | | | | |
|---|---|---|---|---|
| Na+/glucose cotransporter | 5′- CAGAGACCCCATTACTGGAGAC | 1 | 5′- TCGTTGCACAATGACCTGATC | 14 |
| (SOLT1) | A | | | |
| colipase | 5′- TGACACCATCCTGGGCATT | 2 | 5′- ACACCGGTAGTAAATCCCATAA | 15 |
| | | | AGG | |
| liver fatty acid binding protein (L- | 5′- CTCCGGCAAGTACCAATTGC | 3 | 5′- TGTCCTTCCCTTTCTGGATGAG | 16 |
| FABP) | | | | |
| metallothioneinI(MT-I) | 5′- ATGTGCCCAGGGCTGTGT | 4 | 5′- AACAGGGTGGAACTGTATAGGA | 17 |
| | | | AGAC | |
| polymeric immunoglobulin receptor | 5′- CTTCCCTCCTGTCCTCAGAGGT | 5 | 5′- GGCGTAACTAGGCCAGGCTT | 18 |
| (pIgR) | | | | |
| decay accelerating factor (DAF) | 5′- CAACCCAGGGTACAGGCTAGTC | 6 | 5′- GGTGGCTCTGGACAATGTATTTC | 19 |
| small proline-rich protein 2a | 5′- CCTTGTCCTCCCCAAGCG | 7 | 5′- AGGGCATGTTGACTGCCAT | 20 |
| (sprr2a) | | | | |
| multi-drug resistance protein | 5′- GCCGCTTCTTCCAAAGTCTACA | 8 | 5′- CGTGTCTCTACTCCCGGTTTCC | 21 |
| (mdr1a) | | | | |
| glutathione S-transferase (GST) | 5′- CATCCAGCTCCTAGAAGCCATT | 9 | 5′- GGGTTGCAGGAACTTCTTAATTG | 22 |
| | | | TA | |
| lactase-phlorizin hydrolase | 5′- TTGAATGGGCCACAGGCT | 10 | 5′- AGCGGACTATGGAGGCGTAG | 23 |
| adenosine deaminase (ADA) | 5′- GCGCAGTAAAGAATGGCATTC | 11 | 5′- CTGTCTTGAGGATGTCCACAGC | 24 |
| angiogenin-4 | 5′- TCGATTCCAGGTCACCACTTG | 12 | 5′- CACAGGCAATAACAATATATCT | 25 |
| | | | GAAATCT | |
| glyceraldehyde 3-phosphate | 5′- TGGCAAAGTGGAGATTGTTGCC | 13 | 5′- AAGATGGTGATGGGCTTCCCG | 26 |
| dehydrogenase | | | | |

Each cRNA was hybridized to Affymetrix Mu11K and Mu19K chip sets representing ˜25,000 unique mouse genes from Unigene Build 4 and the TIGR cluster databases, according to Affymetrix protocols. Data collected from each chip were scaled so that the overall fluorescence intensity across each chip was equivalent (target intenstity=150). Pairwise comparisons of ‘germ-free’ versus ‘colonized’ expression levels were performed.

A 2-fold or more difference was recorded if three criteria were met: the GeneChip software returned a difference call of “increased” or “decreased”, the mRNA was called ‘present’ by GeneChip software in either germ-free or colonized cRNA, and the difference was observed in duplicate microarray hybridizations.

mRNAs represented by 118 probe sets changed by at least 2-fold with colonization, as defined by duplicate microarray hybridizations.

It was found that transcripts represented by 95 probe-sets were increased, while those represented by 23 probe-sets were decreased. The genes represented by 84 of these probe sets (71 unique genes) were assigned to functional groups and these are set out in Table 1 hereinafter. In this table, results are presented as the fold-difference in mRNA levels between colonized and germ-free ileum and represent average values from duplicate microarray hybridizations. The average fold-changes for genes represented by 2 or more independent probe sets are listed separately.

TABLE 1
Colonization-associated changes in distal small intestinal gene
expression

| | | GenBank/TIGR | average |
|---|---|---|---|
| Gene | function | Reference | fold Δ |
| Nutrient Uptake and | | | |
| Metabolism | | | |
| carbohydrates | | | |
| Na+/glucose | glucose uptake | AF163846 | +2.4 |
| cotransporter (SGLT1) | | | |
| lactase phlorizin- | lactose | AA521747 | −2.2 |
| hydrolase | hydrolysis | | |
| lipids | | | |
| pancreatic lipase- | lipid metabolism | M30687 | +4.1 |
| related protein 2 | | | |
| colipase | lipid metabolism | AA611440 | +9.4 |
| liver fatty acid | lipid metabolism | Y14660 | +4.0, |
| binding protein | | | +5.6 |
| apolipoprotein A-IV | lipid metabolism | M13966 | +2.2 |
| fasting-induced | regulation of | AF278699 | −9.0 |
| adipose factor | lipid metabolism | | |
| phospholipase B | lipid metabolism | TC38683 | −2.2 |
| CYP27 | cholesterol 27- | TC25974 | −2.2 |
| | hydroxylation | | |
| metals | | | |
| high-affinity copper | copper uptake | AA190119 | +2.6 |
| transporter | | | |
| metallothionein I | Cu/Zn | V00835 | −4.6, |
| | sequestration | | −6.1 |
| metallothionein II | Cu/Zn | K02236 | −5.7, |
| | sequestration | | −6.3 |
| ferritin heavy chain | iron | M24509 | −4.5 |
| | sequestration | | |
| cellular energy | | | |
| production | | | |
| isocitrate | citric acid | U68564 | +2.4 |
| dehydrogenase subunit | cycle | | |
| cytochrome c oxidase | mitochondrial | TC106691 | +2.4 |
| subunit 1 | electron | | |
| | transport | | |
| succinyl CoA | ketone body | TC18674 | +2.0 |
| transferase | utilization | | |
| transketolase | pentose | u05809 | +2.4 |
| | phosphate | | |
| | pathway | | |
| phosphogluconate | Pentose | C81475 | +2.8 |
| dehydrogenase | phosphate | | |
| | pathway | | |
| malate oxidoreductase | malate-aspartate | J02652 | +6.0 |
| | shuttle | | |
| aspartate | malate-aspartate | J02623 | +2.5 |
| aminotransferase | shuttle | | |
| hormonal/maturational | | | |
| responses | | | |
| adenosine deaminase | adenosine | M10319 | +2.3 |
| | inactivation | | |
| omithine decarboxylase | regulation of | U52823 | +2.4 |
| antizyme | polyamine levels | | |
| 15- | prostaglandin | U44389 | −3.2 |
| hydroxyprostaglandin | inactivation | | |
| dehydrogenase | | | |
| GARG-16 | response to | U43084 | −4.0, |
| | glucocorticoid | | −4.5 |
| | production | | |
| FKBP51 | component of | U16959 | −3.8 |
| | steroid receptor | | |
| | complex | | |
| androgen-regulated vas | steroidogenesis | J05663 | −3.3, |
| deferens protein | | | −3.4 |
| short chain | steriod/retinoid | AF056194 | −2.2, |
| dehydrogenase | metabolism | | −2.8 |
| heat-stable antigen | hematopoietic | X53825 | +3.0 |
| | differentiation | | |
| | marker | | |
| Mucosal barrier | | | |
| function | | | |
| decay-accelerating | complement | D63679 | +5.2 |
| factor | inactivation | | |
| polymeric Ig receptor | transepithelial | U06431 | +2.3 |
| | IgA transport | | |
| small proline-rich | crosslinking | AJ005559 | +10.6, |
| protein 2a | protein | | +102 |
| serum amyloid A | acute phase | U60437 | +2.8, |
| protein | response | | +5.4 |
| CRP-ductinα (MUCLIN) | mucin-like | U37438 | +2.4 |
| | protein | | |
| zeta proteasome chain | antigen | AF019661 | +2.8 |
| | presentation | | |
| anti-DNA IgG light | | U55583 | +2.5 |
| chain | | | |
| Detoxification/drug | | | |
| resistance | | | |
| glutathione S- | GSH conjugation | L06047 | −2.4 |
| transferase | to electrophiles | | |
| P-glycoprotein (mdr1a) | export of GSH- | M33581 | −4.6 |
| | conjugated | | |
| | compounds | | |
| CYP2D2 | 4-hydroxylase | TC36686 | −2.6 |
| Enteric nervous | | | |
| system/muscular layers | | | |
| L-glutamate | glutamate uptake | U73521 | +4.4 |
| transporter | | | |
| L-glutamate | GABA production | M55253 | +2.2 |
| decarboxylase | | | |
| vesicle-associated | neurotransmitter | AF157497 | +2.2 |
| protein-33 | release | | |
| cysteine-rich protein 2 | cGMP kinase I | AA028770 | +3.2 |
| | target | | |
| smooth muscle | contractility | M26689 | +2.3 |
| (enteric) gamma actin | | | |
| SM-20 | growth-factor | TC33445 | +4.8 |
| | responsive gene | | |
| Calcium channel5 | calcium channel | AJ272046 | −2.2 |
| subunit | regulation | | |
| angiogenesis | | | |
| angiogenin-4 | unknown | SEQ ID NO | +10.9 |
| | | 29 | |
| angiogenin-related | unknown | U22519 | +6.4 |
| protein | | | |
| angiogenin family¹ | | | +2.4, |
| | | | +6.0, |
| | | | +7.0 |
| cytoskeleton/extra- | | | |
| cellular matrix | | | |
| gelsolin | actin binding | J04953 | +7.9 |
| | protein | | |
| destrin | actin | W17549 | +3.0 |
| | depolymerizing | | |
| | factor | | |
| alpha cardiac actin | contractility | M15501 | +3.4 |
| endoB cytokeratin | intermediate | m11686 | +3.0 |
| | filament protein | | |
| fibronectin | extracellular | M18194 | +2.9, |
| | matrix protein | | +3.2 |
| proteinase inhibitor 6 | serine protease | U25844 | +2.6 |
| | inhibitor | | |
| mpgc60 | serine protease | Y11505 | +2.5 |
| | inhibitor | | |
| alpha 1 type 1 | extracellular | X06753 | +2.2, |
| collagen | matrix protein | | +4.7 |
| signal transduction | | | |
| Pten | protein/lipid | U92437 | +3.2 |
| | phosphatase | | |
| gp106 (TB2/DP1) | unknown | U28168 | +6.9 |
| rac2 | ras-related GTP- | X53247 | +7.0 |
| | binding protein | | |
| Semcap2 | SemaF-associated | AF061262 | −2.9 |
| | protein | | |
| serum and | serine/threonine | AF139638 | −2.6 |
| glucocorticoid- | protein kinase | | |
| regulated kinase | | | |
| STE20-like protein | serine/threonine | AA154321 | +2.6 |
| kinase | protein kinase | | |
| B-cell myeloid kinase | unkown | J03023 | +2.1 |
| general cellular | | | |
| functions | | | |
| glutathione reductase | maintainance of | X76341 | +2.9 |
| | reduced | | |
| | glutathione | | |
| calmodulin | calcium | M27844 | +2.2 |
| | homeostasis | | |
| elF3 subunit | translation | U70736 | +2.7 |
| | initiation | | |
| hsc70 | stress response | U73744 | +2.9 |
| oligosaccharyl | protein N- | U84211 | +3.4 |
| transferase subunit | glycosylation | | |
| fibrillarin | ribosomal RNA | Z22593 | +2.4 |
| | processing | | |
| H+-transporting ATPase | intracellular | AA108559 | +2.9 |
| | organelle | | |
| | acidification | | |
| Msec23 | component of the | AA116735 | +2.8 |
| | COPII complex | | |
| vacuolar protein | membrane protein | U47024 | +2.4 |
| sorting 35 | recycling | | |

Importantly, a large of number of the genes identified using these criteria are involved in modulating fundamental intestinal functions: 20 of the 71 genes (28%) were grouped under nutrient uptake and metabolism. There was also a concerted rise in expression of several components of the host's lipid absorption/export machinery, including pancreatic lipase-related protein-2 (PLRP-2), colipase, liver fatty acid binding protein (L-FABP), and apolipoprotein A-IV (Table 1). The prominent. decrease in expression of fasting-induced adipose factor, a novel PPARg target known to be repressed with fat feeding (S. Kersten, et al., J. Biol. Chem. 275, 28488 (2000), provided further evidence for augmented lipid uptake in colonized mice. The changes in expression of these 6 genes indicate that B. thetaiotaomicron elicits an increased host capacity for nutrient absorption/processing and helps explain why germ-free rodents require a higher caloric intake to maintain their weight than those with a microflora.

Additionally, there were changes in expression of four genes involved in dietary metal absorption. A high affinity epithelial copper transporter (CRT1) mRNA was increased, while metallothionein-I, metallothionein-II, and ferritin heavy chain mRNAs were decreased (Table 1). These changes suggest that colonization engenders increased capacity to absorb heavy metals (e.g., via CRT1) and a concomitant decreased capacity to sequester them within cells (MT-I/II, ferritin). This implies greater host demand for these compounds, either due to increased utilization by the host's own metabolic pathways or to competition with the microbe. The changes in SGLT-1, colipase, L-FABP, and MT1 (plus 8 other mRNAs discussed below), were independently validated by qRT-PCR (C. A. Heid, J. Stevens, K. J. Livak, P. M. Williams, Genome Res. 6, 986 (1996). (Table 2).

Of these, genes which were found to have a difference in expression levels of 5-fold or more as a result of B. thetaiotaomicron colonisiation were colipase, liver fatty acid binding protein, fasting-induced adipose factor, metallothionein I and metallothionein II, malate oxidoreductase, Sprr2a, angiogenin-3, angiogenin-related protein, angiogenin family, gelsolin, gp106(TB2/DP1) and rac 2. Of these, colipase, fasting-induced adipose factor, angiogenin 3 and Sprr2a genes showed a difference in expression levels of 9-fold or more.

A notable feature of the host response to B. thetaiotaomicron was the absence of detectable or changed expression of the many genes involved in immuno-inflammatory processes that are represented on the microarrays. These include genes involved in the NF-κB-regulated processes that are critical regulators of host responses to invasive pathogens (D. Elewaut et al., J. Immunol. 163, 1457 (1999)). The absence of these responses can be contrasted to results obtained in a recent cDNA microarray analysis of the response of a human intestinal epithelial cell line to Salmonella, an invasive gut pathogen (L. Eckmann, J. R. Smith, M. P. Housley, M. B. Dwinell, M. F. Kagnoff, J. Biol. Chem. 275, 14084 (2000)). The lack of evidence for an evoked in vivo immuno-inflammatory response is consistent with the host's need to accommodate resident gut microbes, such as B. thetaiotaomicron, for its entire lifespan.

Colonization increases expression of two genes implicated in development of gut neoplasia, Pten and Gp106 (Table 1).

### EXAMPLE 2

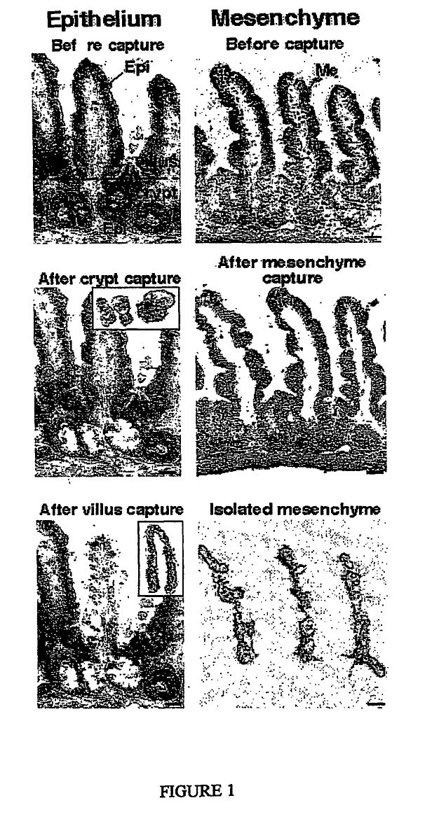

In a further analysis two techniques were combined. First, laser-capture microdissection (LCM) was used to recover three cell populations from frozen sections of ileum harvested immediately after sacrifice of germ-free and colonized mice. The three populations are (i) epithelium present in crypts (the proliferative compartment of the intestine containing undifferentiated cells as well as differentiated members of the Paneth cell lineage); (ii) epithelium overlying villi (containing post-mitotic, differentiated members of the intestine's other three lineages); and (iii) mesenchyme underlying crypt-villus units (FIG. 1).

LCM was performed on groups of mice independent of those used to generate RNA for the microarray analysis. 7 μm-thick sections were cut from frozen ileums and LCM conducted using the PixCell II system from Arcturus (7.5 μm diameter laser spot). RNA was prepared from dissected cell populations using the RNA Micro-Isolation Kit (Strategene) and standard histochemical protocols. Laser capture microdissection (LCM) was carried out using conventional methods as described by M R Emmert-Buck et al., Science., 274, 998-1001, 1996 and R. F. Bonner et al., Science 278:1203-4, 1997.

The results are shown in FIG. 1.

Second, real-time RT-PCR was used to quantitate levels of specific mRNAs in the laser captured cell populations. The LCM/qRT-PCR analysis was performed using germ-free and colonized mice from 3 experiments that were independent of those used for microarray profiling.

Each sample was analyzed in triplicate in 4-independent experiments. Mean values for the independent determinations ±1 S. D. are shown in Table 2

TABLE 2
Real-time quantitative RT-PCR studies of colonization-
associated changes in gene expression

| | Fold Δ |
|---|---|
| | (relative to |
| Gene | germ-free) |
| Na+/glucose cotransporter (SGLT1) | 2.6 ± 0.9 |
| colipase | 6.6 ± 1.9 |
| liver fatty acid binding protein (L-FABP) | 4.4 ± 1.4 |
| metallothionein I (MT-I) | −5.4 ± 0.7 |
| polymeric immunoglobulin receptor (pIgR) | 2.6 ± 0.7 |
| decay accelerating factor (DAF) | 5.7 ± 1.5 |
| small proline-rich protein 2a (sprr2a) | 205 ± 64 |
| multi-drug resistance protein (mdr1a) | −3.8 ± 1.0 |
| glutathione S-transferase (GST) | −2.1 ± 0.1 |
| lactase-phlorizin hydrolase | −4.1 ± 0.6 |
| adenosine deaminase (ADA) | 2.6 ± 0.6 |
| angiogenin-4 | 9.1 ± 1.8 |

Colipase is produced by the exocrine (acinar) cells of the pancreas. Expression in the intestine had not been reported previously. Therefore, LCM and real-time RT-PCR analysis were employed to delineate the cellular origins of its response to B. thetaiotaomicron.

The results show that sprr2a mRNA is confined to the epithelium where its concentration is 7-fold higher on the villus compared to the crypt (FIG. 1B). B. thetaiotaomicron elicits a 280-fold increase in the villus epithelium. This value is in good agreement with the increase documented in total ileal RNA (Table 2). The cellular origin of the sprr2a response supports the hypothesis that it participates in fortifying the intestinal epithelial barrier in response to bacterial colonization.

Colipase is produced by the exocrine acinar cells of the pancreas. LCM/qRT-PCR revealed that colipase mRNA is also present in the ileal crypt epithelium, where it increases 10-fold upon B. thetaiotaomicron colonization (FIG. 1B). This accounts for the increase detected by microarray and qRT-PCR analyses of total ileal RNA (Tables 1,2). Colipase plays a critical role in dietary lipid metabolism by stimulating the activity of both pancreatic triglyceride lipase and PLRP-2 (M. E. Lowe, et al., J. Biol. Chem. 273, 31215 (1998). Furthermore, proteolytic cleavage of procolipase yields a pentapeptide (enterostatin) that functions as a satiety signal for fat ingestion (S. Okada, et al., Physiol. Behav. 49, 1185 (1991). Analyses of colipase gene regulation reveal a previously unappreciated contribution of the intestinal epithelium (together with a resident gut commensal) to dietary lipid metabolism.

Angiogenin-3 was originally identified in NIH 3T3 fibroblasts (X. Fu et al., Mol. Cell Biol. 17, 1503 (1997), but little is known about its cellular origins or regulation. LCM and qRT-PCR revealed that the crypt epithelium is the predominant location of a gene amplifiable using primers such as SEQ ID NO 12 and 25 (see Table 3 hereinbefore) which are specific for angiogenin-3 mRNA, but also for the new protein which is angiogenin-4 mRNA and that colonization results in a 7-fold increase in its levels within this compartment. This increase accounts for the change in expression defined by microarray and qRT-PCR analyses of total ileal RNA (Tables 1,2). The epithelial location of a secreted/RNAse/angiogenesis factor puts it in a strategic position to function as an effector of a number of host responses to microbial colonization (e.g., enhanced absorption/distribution of nutrients/augmented barrier function.

The LCM/qRT-PCR studies of sprr2a, colipase and angiogenin-4 establish the feasibility of assigning an in vivo host response to a particular cell population in a complex tissue, and of describing the cellular response in quantitative terms. In recovering a responding cell population and expressing its reaction to a microorganism in quantitative terms, the applicants results demonstrate how it is possible to move beyond in vitro models and use in vivo systems to study the impact of a microbe on host cell gene expression.

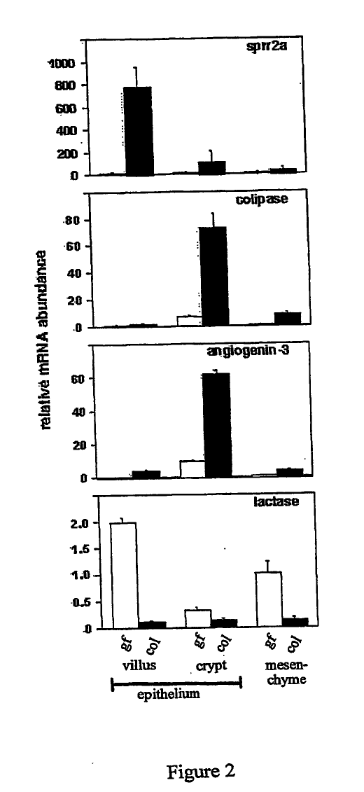

Colonization of germ-free mice with B. thetaiotaomicron produces a decrease in ileal LPH mRNA levels (Table 1,2) (although not by as much as five times). Analysis of RNA isolated from laser-captured epithelial and mesenchymal cell populations established that the colonization-induced reduction in LPH mRNA levels occurs primarily within the villus epithelium (FIG. 2).

Comparison of transcript levels between germ-free and B. thetaiotaomicron-associated mice revealed a colonization-associated increase in expression of angiogenin-4.

The 11-fold induction of its mRNA seen in Example 1 was independently validated by real-time RT-PCR of total ileal RNAs (Table 2). Angiogenin-3 was originally identified in NIH 3T3 fibroblasts (X. Fu, M. P. Kamps, Mol. Cell Biol. 17, 1503 (1997)). However, LCM and real-time RT-PCR analysis revealed that in colonized ileum, the levels of mRNA which are amplifiable using primers designed for angiogenin-3 are highest in crypt epithelium (values in the ileal villus epithelium and mesenchyme are 14- and 15-fold lower, respectively; FIG. 2). As outlined in Example 4 below however, this mRNA is in fact, angiogenin-4 mRNA.

The 7-fold increase in these angiogenin-4 mRNA levels observed in the crypt epithelium after colonization account for the change defined by microarray and real-time RT-PCR analyses of total ileal RNA.

Bacterial modulation of epithelially-expressed angiogenin-4 represents a novel mode of regulation for an angiogenesis factor.

### EXAMPLE 3

LCM/qRT-PCR established that colonization reduces lactase mRNA levels within the villus epithelium (FIG. 1B). The concept that microbes may help legislate changes in expression of developmentally-regulated genes, such as lactase, raises the question of whether some or many components of the microflora can elicit these changes.

In order to examine this, age-matched groups (n=4-8 mice/group) of 7-15 week-old germ-free NMRI/KI mice were maintained in plastic gnotobiotic isolators on a 12 hour light cycle, and given free access to an autoclaved chow diet (B&K Universal). Males were inoculated with one of the following groups

(i) Nothing—Germ-free control,

(ii) B. thetaiotaomicron strain VPI-5482 (L. V. Hooper, et al., Proc. Natl. Acad. Sci. U.S.A. 96, 9833 (1999)),

(iii) E. coli K12 which was originally recovered from a normal human fecal flora, (iv)Bifidobacterium infantis (ATCC15697), a prominent component of the pre-weaning human and mouse ileal flora and a commonly used probiotic.

(v) a ‘complete’ ileal/cecal microflora harvested from conventionally-raised mice (L. Bry, et al., Science 273, 1380 (1996)

A further control group comprised mice conventionally raised since birth.

Mice were sacrificed 10 days later, 2 hours after lights were turned on. The distal 1 cm of the small intestine was used to define CFU/ml ileal contents. The 3 cm of intestine just proximal to this segment was used to isolate total ileal RNA (Qiagen RNeasy kit).

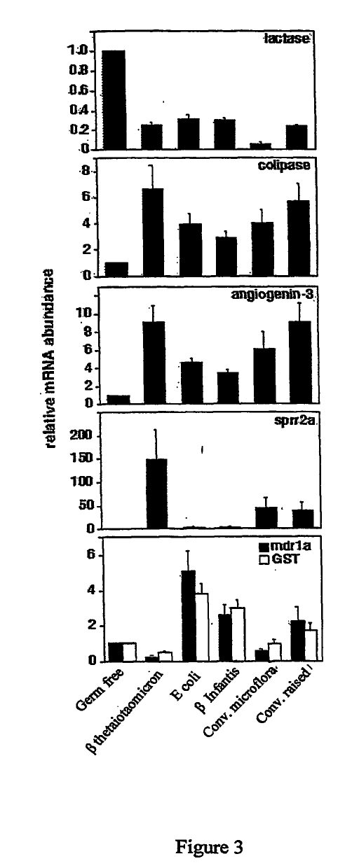

qRT-PCR was used to compare ileal lactase mRNA levels in each group (all animals had =10⁷ CFU/ml ileal contents). The results are shown in FIG. 3.

Colonization with any of the three gram-negative anerobes elicited an equivalent decline in lactase expression relative to germ-free controls (FIG. 3). This decline was also observed after inoculation of a complete ileal/cecal flora. qRT-PCR of the same RNAs revealed that ileal expression of colipase and angiogenin-4 was induced after colonization of all three organisms, and by the ileal/cecal flora (FIG. 3).

The levels of colipase and angiogenin-4 mRNAs achieved in the ileums of these ex-germ-free mice were comparable to those of age-matched mice that have been conventionally-raised since birth (FIG. 3).

In contrast to these findings, the response of sprr2a to colonization was dependent upon the colonizing species. While B. thetaiotaomicron produced a pronounced rise in sprr2a mRNA that recapitulates the response to a 10 day colonization with the ileal/cecal flora, colonization with B. infantis and E. coli produce only negligible increases in mRNA levels (FIG. 3).

Mdr1a and glutathione-S-transferase, which act in concert to metabolize xenobiotics and electrophiles, also exhibited species-specific (and concerted) responses. Unlike B. thetaiotaomicron, which suppresses expression, E. coli and B. infantis both elicit increases in these mRNAs. In contrast, the multi-component ileal/cecal flora did not produce a significant (i.e.,=2-fold) change in levels of either mRNA when compared to germ-free controls.

The Mdr1a/GST responses provide direct evidence that components of the normal microflora can modulate host genes involved in drug metabolism, and suggest that variations in drug metabolism between individuals may arise, in part, from differences in their resident gut flora.

### EXAMPLE 4

Following the observation that a 10 d colonization was associated with a 11-fold increase in ileal expression of a mRNA detected by an Affymetrix-designed probe-set designed from the published sequence of angiogenin-3, we designed primers specific for the 3′ and 5′ ends of the mouse angiogenin-3. There were:

ORF [forward primer:

5′-CCTTGGATCCATGGTGATGAGCCCAGGT TCTTTG (SEQ ID NO 27)

which incorporates a BamHI site at the 5′ end;

reverse primer:

5′-CCTTTCTAGACTACGGACTGATAAAAGACTCATCGAAG (SEQ ID NO 28)

which incorporates an XbaI site at the 5′ end.

These primers were used together with RT-PCR to amplify a 438 bp sequence from RNA prepared from the ileums of ex-germ-free NMRI mice. These mice had been colonized for 10 d with a complete ileal/cecal flora harvested from conventionally-raised animals belonging to the same inbred strain. We subcloned the PCR product into BamHI/XbaI digested pGEX-KG and sequenced it using vector-specific primers.

Surprisingly, the nucleotide sequence of the ORF was only 90% identical to that of mouse angiogenin-3. Since the primer sequences used in the PCR reaction (specific for angiogenin-3) were incorporated into the product, we used 5′- and 3′-RACE to (a) obtain accurate sequence at the 5′ and 3′ ends of the ORF of this new angiogenin, and (b) characterize the 5′- and 3′ untranslated regions of its mRNA. The results revealed only 88.3% nucleotide sequence identity with angiogenin-3 mRNA.

The nucleotide sequence which encodes the angiogenin-4 protein, aligned with the angiogenin-3 sequence is shown hereinafter in FIG. 4 as SEQ ID NO 29 and 30 respectively.

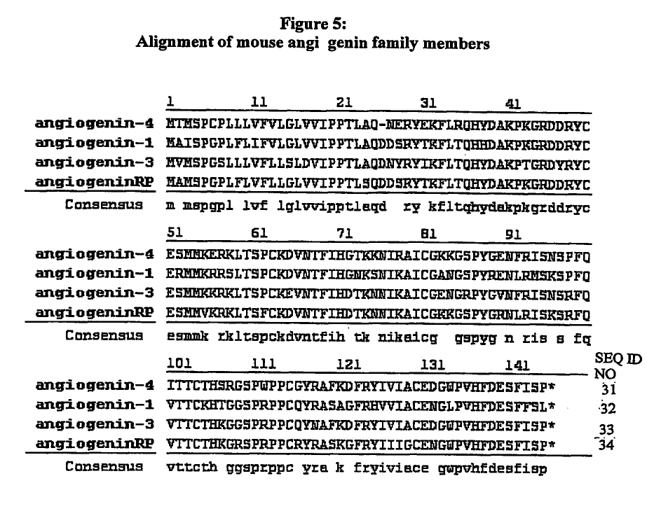

Angiogenin-4 has 74 to 81% amino acid sequence identity to the other 3 members of the mouse angiogenin family (FIG. 5). It was found that the 5′ and 3′-untranslated regions of angiogenin-4 are closely related to the corresponding regions of angiogenin-3 mRNA (FIG. 4).

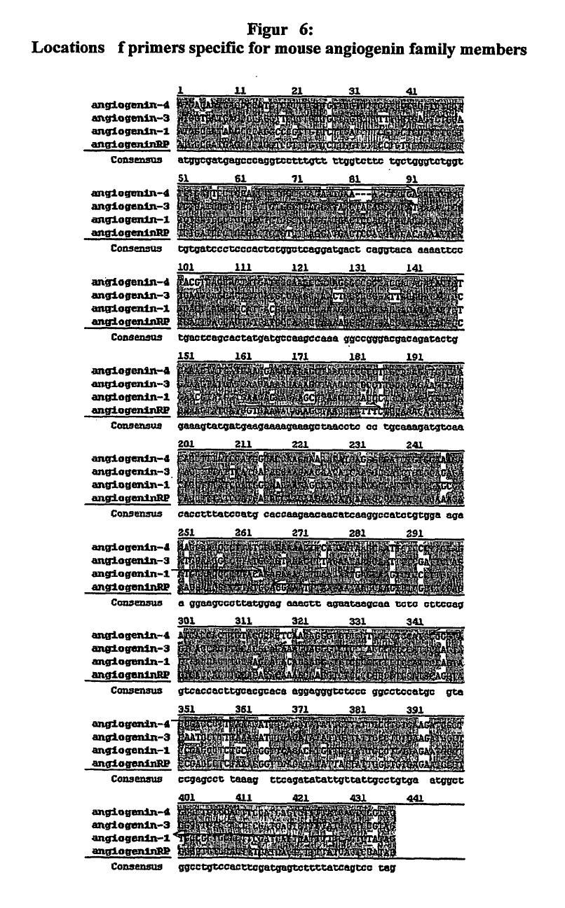

Subsequently a comparative analysis of the tissue distribution of the various mouse angiogenin mRNAs, was conducted. cDNA was synthesized from RNAs isolated from tissues harvested from conventionally raised adult (12-14 week old) male and female NMRI mice (25 tissues/mouse). To quantitate relative levels of expression of each gene, we designed primer sets specific for each of the four mouse angiogenin family members (FIG. 6; Table 4) and used them for SYBR-Green-based real-time quantitative RT-PCR (qRT-PCR) analyses.

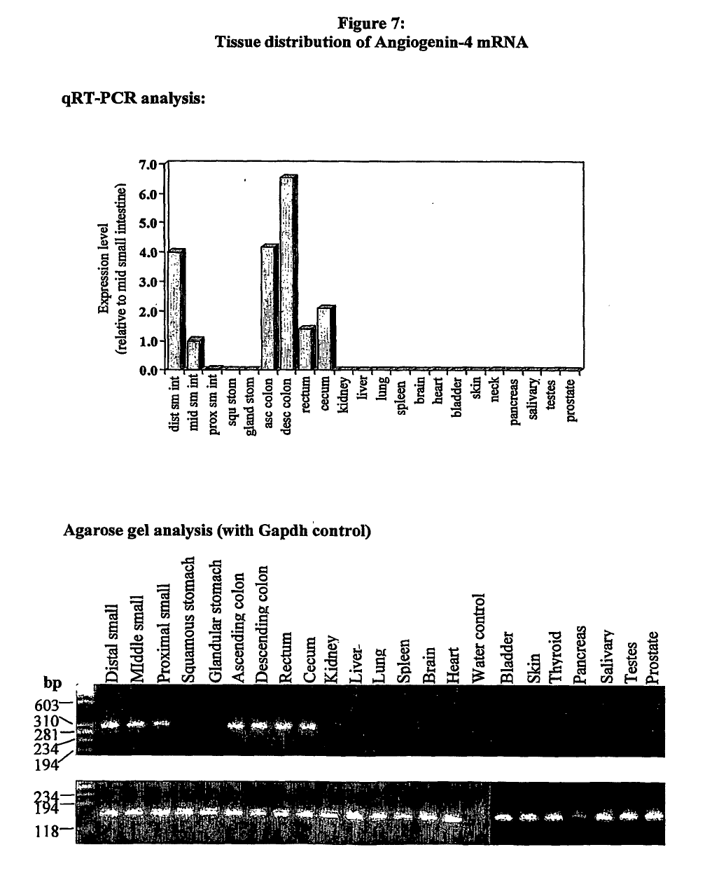

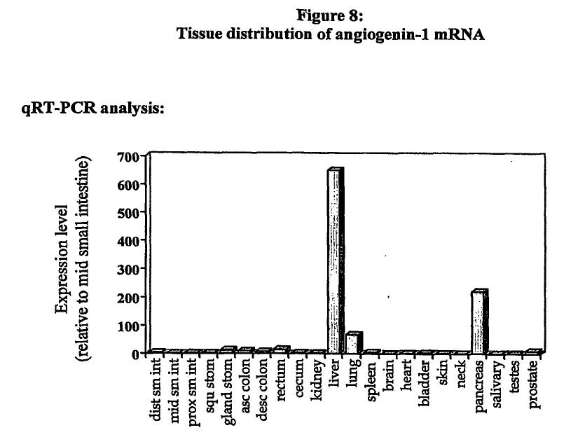

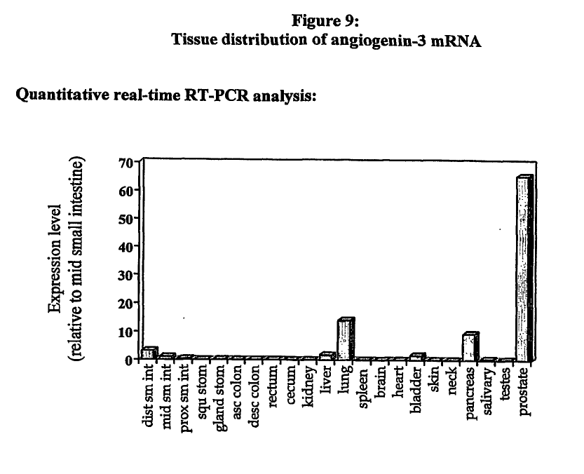

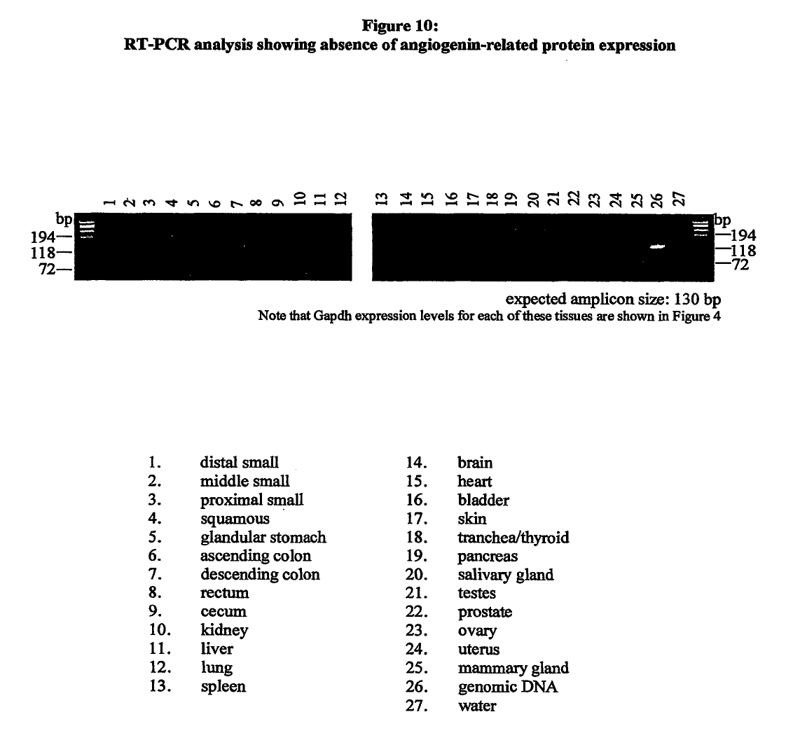

Remarkably, angiogenin-4 mRNA was restricted the intestine where it is expressed from the duodenum to the rectum (FIG. 7). In contrast, angiogenin-1 expression is highest in liver, lung, and pancreas (FIG. 8), while angiogenin-3 is expressed primarily in liver, lung, pancreas, and prostate (FIG. 9). Angiogenin-related protein mRNA was undetectable in all tissues surveyed even after 40 cycles of PCR (FIG. 10).

Thus, the highly restricted, intestine-specific pattern of angiogenin-4 expression makes it unique among mouse angiogenin family members.

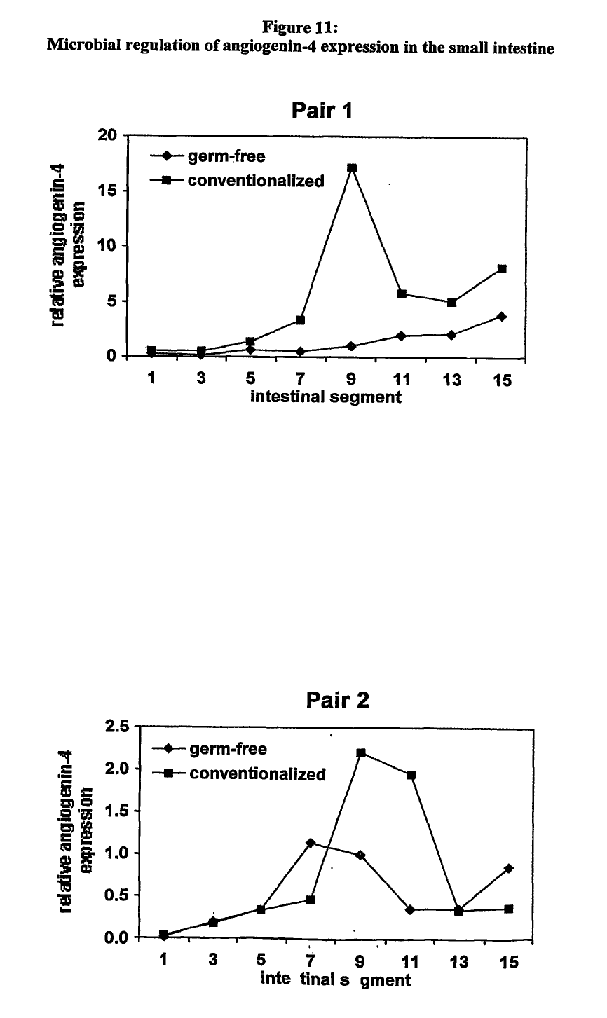

These findings indicated that there was microbial-regulation of angiogenin-4 rather than angiogenin-3 expression in the intestine. To test this hypothesis directly, angiogenin-4-specific primers and qRT-PCR were used to compare angiogenin-4 mRNA levels along the length of the small intestine of germ-free NMRI mice and germ-free mice colonized for 10 d with an ileal/cecal flora harvested from conventionally raised NMRI animals. Pair-wise comparisons revealed that expression of angiogenin-4 is highest in the jejunum of colonized mice, and that conventionalization induces up to a 17-fold increase in angiogenin-4 expression in this region (FIG. 11). Mono-association of germ-free NMRI mice with B. thetaiotaomicron for 10 d resulted in a comparable induction of angiogenin-4 expression (data not shown).

TABLE 4

| | | | |
|---|---|---|---|
| | | SEQ | |
| | | ID | |
| Gene | Primer | NO | Sequence |

| | | | |
|---|---|---|---|
| angio- | forward | 35 | 5′CTCTGGCTCAGAATGTAAGGTACGA |
| genin-4 | | | |
| | reverse | 36 | 5′GAAATCTTTAAAGGCTCGGTACCC |
| angio- | forward | 37 | 5′CTGGCTCAGGATAACTACAGGTACAT |
| genin-3 | | | |
| | reverse | 38 | 5′GCCTGGGAGACCCTCCTTT |
| angio-1 | forward | 39 | 5′AGCGAATGGAAGCCCTTACA |
| genin-1 | | | |
| | reverse | 40 | 5′CTCATCGAAGTGGACCGGCA |
| angio- | forward | 41 | 5′GGTGAAAAGAAAGCTAACCTCTTTC |
| genin- | | | |
| related protein | reverse | 42 | 5′AGACTTGCTTATTCTTAAATTTCG |

Regulation of Angiogenin-4 Expression During Postnatal Development is Consistent with its Microbial Regulation

The developmental patterns of angiogenin-4 expression in postnatal day 5 (P5)—P30 germ-free and conventionally raised NMRI mice (n=3 mice per time point per group) was then assessed (FIG. 9). Relative levels of the angiogenin-4 transcript remained relatively low until P20 in both groups of mice. Expression rose slightly (2-3 fold) in germ-free animals after this time point. In contrast, angiogenin-4 expression increased more than 20-fold between P15 and P30 in conventionally-raised animals. These results indicate that angiogenin-4 is induced during the suckling/weaning transition —coincident with a major shift in the gut microbiota. The lack of angiogenin-4 induction in postnatal germ-free mice is also consistent with the conclusion that components of the microbiota play an important role in regulating angiogenin-4 expression.

Cellular Localization of Angiogenin-4

The previous laser capture microdissection (LCM)/qRT-PCR study of the cellular origins of angiogenin protein expression (Example 2) used primers that recognize both angiogenin-3 and angiogenin-4, and RNAs that had been isolated from captured crypt epithelium, villus epithelium, or mesenchymal populations from the villus core. The qRT-PCR analysis indicated that the microbially-regulated ‘angiogenin’ was produced in epithelial cells located at the base of crypts of Lieberkuhn (Hooper et al., 2001).

To test the hypothesis that angiogenin-4 expression occurs in Paneth cells, we used LCM to isolate cells located at the base of jejunal crypts from (a) germ-free adult (12 week old) transgenic mice with an attenuated diphtheria toxin-A fragment (tox176)-mediated Paneth cell lineage ablation (CR2-tox176 mice) (Garabedian et al., 1997), and (b) their age and gender-matched germ-free normal littermates. qRT-PCR using angiogenin-4-specific primers revealed that angiogenin-4 mRNA levels are 10-fold higher in RNA purified from crypt base epithelial cells of normal mice compared to CR2-tox176 littermates (FIG. 10).

A follow-up study was conducted using conventionally raised NMRI mice. Three cellular pools were harvested by LCM: Paneth cells alone, epithelial cells from the upper crypt and villus (a Paneth cell-minus fraction), and mesenchyme retrieved from the villus core and the peri-cryptal region. The distribution of angiogenin-4 mRNA closely paralleled the distribution of phospholipase A2—the product of the Mom-1 locus and a well-known Paneth cell-specific gene product (data not shown).

1. A method of investigating chemical changes resulting from commensal microflora colonisation of mammalian intestine which comprises:
a) measuring gene expression in commensal bacterium-colonized and germ-free intestine of at least one gene; and
b) identifying a gene from a) that has at least a 2-fold difference in expression level between commensal bacterium-colonized and germ-free intestine.

2. A method according to claim 1 wherein in step (a) multiple gene expression is measured by DNA microarray analysis and/or quantitative RTPCR.

3. A method according to claim 1 which further comprises the step of c) investigating a gene identified in b) with regard to its function, in a system selected from the group consisting or in vitro cell culture, lower eukaryotic model organisms or an animal model.

4. A method according to claim 3 wherein the function is studied using a method selected from the group consisting of
i) transgenic knockout;
ii) dominant-negative experiments;
iii) transgene overexpression;
iv) antibody binding assay;
v) by pharmacological intervention using defined chemical agents.

5. A method according to claim 1 wherein the commensal bacteria is B. thetaiotaomicron.

6. A method according to claim 1 wherein expression of at least 10 genes is measured and said genes are selected from genes associated with the nutrient uptake and metabolism, hormone/maturational responses, mucosal barrier function, detoxification/drug resistance, enteric nervous system/muscular layer development or activity, angiogenesis, cytoskeleton/extracellular matrix function or development, signal transduction or other cellular functions are measured in step (a).

7. A method according to claim 6 wherein expression of at least 10 genes selected from the group consisting of genes encoding Na+/glucose cotransporter (SGLT1), lactase phlorizin-hydrolase, pancreatic lipase-related protein 2, colipase, liver fatty acid binding protein, fasting induced adipose factor (FIAF), apolipoprotein A-IV, phospholipase B, CYP27 high-affinity copper transporter, metallothionein I, metallothionein II, ferritin heavy chain, isocitrate dehydrogenase subunit, succinyl CoA transferase, transketolase, malate oxidoreductase, aspartate aminotransferase, adenosine deaminase, omithine decarboxylase antizyme, 15-hydroxyprostaglandin dehydrogenase, GARG-16, FKBP51, androgen-regulated vas deferens protein, short chain dehydrogenase, heat-stable antigen, decay-accelerating factor, polymeric Ig receptor, small proline-rich protein 2a, serum amyloid A protein, CRP-ductinα (MUCLIN), zeta proteasome chain, anti-DNA IgG light chain, glutathione S-transferase, P-glycoprotein (mdr1a), CYP2D2, L-glutamate transporter, L-glutamate decarboxylase, vesicle-associated protein-33, cysteine-rich protein 2, smooth muscle (enteric) gamma actin, SM-20, angiogenin-4, gelsolin, destrin, alpha cardiac actin, endoB cytokeratin, fibronectin, proteinase inhibitor 6, alpha 1 type 1 collagen, Pten, gp106 (TB2/DP1), rac2, Semcap2, serum and glucocorticoid-regulated kinase, STE20-like protein kinase and B-cell myeloid kinase, glutathione reductase, calmodulin, elF3 subunit, hsc70, oligosaccharyl transferase subunit, fibrillarin, H+-transporting ATPase and Msec23.

8. A method according to claim 6 wherein said 10 genes include genes encoding colipase, decay-accelerating factor (DAF), the polymeric IgA receptor, small proline-rich protein 2a (Sprr2a), angiogenin-3, angiogenin-4, Pten, CYP2D2, Sprr2a, rac2 and Mdr-1.

9. A method according to claim 7 wherein expression of substantially all of said genes is measured.

10. A method according to claim 1 wherein in step (b) a gene for which at least a 7-fold difference in expression is identified.

11. A method according to claim 3 which comprises evaluation of the biochemical pathway in which angiogenin-4 or colipase participates in the intestine.

12. A method of changing the expression levels of a particular gene within the digestive tract of a mammal for therapeutic or prophylactic purposes, said method comprises altering the density in the gastrointestinal tract of a commensal bacteria identified using a method according to claim 1 as being able to produce the desired change in said expression level.

13. A method according to claim 12 which comprises modulating epithelially-expressed angiogenesis factor using an effective commensal bacteria identified using a method of claim 1.

14. A method according to claim 12 which comprises modifying metabolism using an effective commensal bacteria identified using a method according to claim 1.

15. A method according to claim 12 which comprises modifying epithelial barrier function using an effective commensal bacteria identified using a method according to claim 1.

16. A method of screening compounds having a pharmaceutical application in a gastrointestinal disease, which method comprises assaying the compounds for their ability to modulate the activity of the product of a gene identified using a method according to claim 1.

17. A method of treating or preventing gastrointestinal disease which method comprises administering therapeutically effective amount of a compound which modulates the activity of the product of a gene identified using a method according to claim 1.

18. A method of screening for a compound potentially useful for treatment or prophylaxis of conditions characterized by a defect in intestinal barrier function which comprises assay of the compound for its ability to modulate the activity or amount of small proline-rich protein 2a (sprr2a) or rac2.

19. The use of a compound able to modulate the activity or amount of small proline-rich protein 2a (sprr2a) or rac2 in preparation of a medicament for the treatment or prophylaxis of conditions characterized by a defect in intestinal barrier function.

20. A method of treating or preventing conditions characterized by a defect in intestinal barrier function which method comprises administration of a therapeutically effective amount of a compound which is able to modulate the activity or amount of small proline-rich protein 2a (sprr2a) or rac2.

21. A method for identifying genes that function as regulators of intestinal biology, said method comprising applying the method as claimed in claim 1 and detecting expression genes which have not heretofore been associated with such function.

22. An angiogenin protein encoded by a gene, at least part of which is amplifiable using primers of SEQ ID NO 12 and 25 above, which is expressed in mouse intestine.

23. A protein according to claim 22 wherein the protein is of SEQ ID NO 29 as shown in FIG. 4 hereinafter, or an allelic variant thereof or a protein which has at least 85% amino acid sequence identity with SEQ ID NO 29.

24. A protein according to claim 23 which is of SEQ ID NO 29.

25. A nucleic acid which encodes a protein according to claim 22.


SEQUENCE LISTING (49 sequences)

SEQ ID NO: 1 - DNA, 23 bp, Artificial Sequence
  Description of Artificial Sequence Primer
cagagaccccattactggagaca

SEQ ID NO: 2 - DNA, 19 bp, Artificial Sequence
  Description of Artificial Sequence Primer
tgacaccatcctgggcatt

SEQ ID NO: 3 - DNA, 20 bp, Artificial Sequence
  Description of Artificial Sequence Primer
ctccggcaagtaccaattgc

SEQ ID NO: 4 - DNA, 18 bp, Artificial Sequence
  Description of Artificial Sequence Primer
atgtgcccagggctgtgt

SEQ ID NO: 5 - DNA, 22 bp, Artificial Sequence
  Description of Artificial Sequence Primer
cttccctcctgtcctcagaggt

SEQ ID NO: 6 - DNA, 22 bp, Artificial Sequence
  Description of Artificial Sequence Primer
caacccagggtacaggctagtc

SEQ ID NO: 7 - DNA, 18 bp, Artificial Sequence
  Description of Artificial Sequence Primer
ccttgtcctccccaagcg

SEQ ID NO: 8 - DNA, 22 bp, Artificial Sequence
  Description of Artificial Sequence Primer
gccgcttcttccaaagtctaca

SEQ ID NO: 9 - DNA, 22 bp, Artificial Sequence
  Description of Artificial Sequence Primer
catccagctcctagaagccatt

SEQ ID NO: 10 - DNA, 18 bp, Artificial Sequence
  Description of Artificial Sequence Primer
ttgaatgggccacaggct

SEQ ID NO: 11 - DNA, 21 bp, Artificial Sequence
  Description of Artificial Sequence Primer
gcgcagtaaagaatggcattc

SEQ ID NO: 12 - DNA, 21 bp, Artificial Sequence
  Description of Artificial Sequence Primer
tcgattccaggtcaccacttg

SEQ ID NO: 13 - DNA, 22 bp, Artificial Sequence
  Description of Artificial Sequence Primer
tggcaaagtggagattgttgcc

SEQ ID NO: 14 - DNA, 21 bp, Artificial Sequence
  Description of Artificial Sequence Primer
tcgttgcacaatgacctgatc

SEQ ID NO: 15 - DNA, 25 bp, Artificial Sequence
  Description of Artificial Sequence Primer
acaccggtagtaaatcccataaagg

SEQ ID NO: 16 - DNA, 22 bp, Artificial Sequence
  Description of Artificial Sequence Primer
tgtccttccctttctggatgag

SEQ ID NO: 17 - DNA, 26 bp, Artificial Sequence
  Description of Artificial Sequence Primer
aacagggtggaactgtataggaagac

SEQ ID NO: 18 - DNA, 20 bp, Artificial Sequence
  Description of Artificial Sequence Primer
ggcgtaactaggccaggctt

SEQ ID NO: 19 - DNA, 23 bp, Artificial Sequence
  Description of Artificial Sequence Primer
ggtggctctggacaatgtatttc

SEQ ID NO: 20 - DNA, 19 bp, Artificial Sequence
  Description of Artificial Sequence Primer
agggcatgttgactgccat

SEQ ID NO: 21 - DNA, 22 bp, Artificial Sequence
  Description of Artificial Sequence Primer
cgtgtctctactcccggtttcc

SEQ ID NO: 22 - DNA, 25 bp, Artificial Sequence
  Description of Artificial Sequence Primer
gggttgcaggaacttcttaattgta

SEQ ID NO: 23 - DNA, 20 bp, Artificial Sequence
  Description of Artificial Sequence Primer
agcggactatggaggcgtag

SEQ ID NO: 24 - DNA, 22 bp, Artificial Sequence
  Description of Artificial Sequence Primer
ctgtcttgaggatgtccacagc

SEQ ID NO: 25 - DNA, 29 bp, Artificial Sequence
  Description of Artificial Sequence Primer
cacaggcaataacaatatatctgaaatct

SEQ ID NO: 26 - DNA, 21 bp, Artificial Sequence
  Description of Artificial Sequence Primer
aagatggtgatgggcttcccg

SEQ ID NO: 27 - DNA, 34 bp, Artificial Sequence
  Description of Artificial Sequence Primer
ccttggatccatggtgatgagcccaggttctttg

SEQ ID NO: 28 - DNA, 38 bp, Artificial Sequence
  Description of Artificial Sequence Primer
cctttctagactacggactgataaaagactcatcgaag

SEQ ID NO: 29 - DNA, 722 bp, Mus sp.
gagcttgacaccgaaggaccctgtctccaggagcacacagctagactcgtccccagttggaggaaagctggccagctttggaatcactgttggaagagatgacaatgagcccatgtcctttgttgttggtcttcgtgctgggtctggttgtgattcctccaactctggctcagaatgaaaggtacgaaaaattcctacgtcagcactatgatgccaagccaaagggccgggacgacagatactgtgaaagtatgatgaaggaaagaaagctaacctcgccttgcaaagatgtcaacacctttatccatggcaccaagaaaaacatcagggccatctgtggaaagaaaggaagcccttatggagaaaacttcagaataagcaattctcccttccagatcaccacttgtacgcactcaagagggtctccctggcctccatgcgggtaccgagcctttaaagatttcagatatattgttattgcctgtgaagatggctggcctgtccacttcgatgagtcttttatcagtccgtagacagcaggcccctggcacagacctaggtctgttttctttttatctcccctcacagccatgatcactggttcaccgttcactgtcacgggccagaaaatgaattatctgaaatatacttctcctcatttataatgcacagaaataaagatatctcaaaamccataaaaaaaaaaaaaaaaaaaaaaaaaa

SEQ ID NO: 30 - DNA, 708 bp, Mus sp.
ctctagcttcacaccgcaggaccctgtctccaggagcacgaagctagacacatcccccgttggaggaaagctggccagctttggaatctctgttggaagagatggtgatgagcccaggttctttgttgttggtctttttgctgagtctggatgtgatccctcccactctggctcaggataactacaggtacataaaattcctgactcagcactatgatgccaagccaactggccgggattacagatactgcgaaagtatgatgaagaaaagaaagctaacctcgccttgcaaagaagtcaacacctttattcatgacaccaagaacaacatcaaggccatctgtggagagaatggaaggccttatggagtaaactttagaataagcaattctcgattccaggtcaccacttgcacgcacaaaggagggtctcccaggcctccatgccagtacaatgcctttaaagatttcagatatattgttattgcctgtgaagatggctggcctgtccacttcgatgagtcttttatcagtccgtagacagcaggcccctggcacagacctaggtctgttttctttttatctcccctcacagccatgatcactggttcagcattcactgtcagtggccagaaaatgaattatctgaaatatacttctcctgatttataatgcacagaaataaagatatctcaaaaaccaaaaaaaa

SEQ ID NO: 31 - protein, 144 aa, Mus sp.
MTMSPCPLLLVFVLGLVVIPPTLAQNERYEKFLRQHYDAKPKGRDDRYCESMMKERKLTSPCKDVNTFIHGTKKNIRAICGKKGSPYGENFRISNSPFQITTCTHSRGSPWPPCGYRAFKDFRYIVIACEDGWPVHFDESFISP

SEQ ID NO: 32 - protein, 145 aa, Mus sp.
MAISPGPLFLIFVLGLVVIPPTLAQDDSRYTKFLTQHHDAKPKGRDDRYCERMMKRRSLTSPCKDVNTFIHGNKSNIKAICGANGSPYRENLRMSKSPFQVTTCKHTGGSPRPPCQYRASAGFRHVVIACENGLPVHFDESFFSL

SEQ ID NO: 33 - protein, 145 aa, Mus sp.
MVMSPGSLLLVFLLSLDVIPPTLAQDNYRYIKFLTQHYDAKPTGRDYRYCESMMKKRKLTSPCKEVNTFIHDTKNNIKAICGENGRPYGVNFRISNSRFQVTTCTHKGGSPRPPCQYNAFKDFRYIVIACEDGWPVHFDESFISP

SEQ ID NO: 34 - protein, 145 aa, Mus sp.
MAMSPGPLFLVFLLGLVVIPPTLSQDDSRYTKFLTQHYDAKPKGRDDRYCESMMVKRKLTSFCKDVNTFIHDTKNNIKAICGKKGSPYGRNLRISKSRFQVTTCTHKGRSPRPPCRYRASKGFRYIIIGCENGWPVHFDESFISP

SEQ ID NO: 35 - DNA, 25 bp, Mus sp.
ctctggctcagaatgtaaggtacga

SEQ ID NO: 36 - DNA, 24 bp, Mus sp.
gaaatctttaaaggctcggtaccc

SEQ ID NO: 37 - DNA, 26 bp, Mus sp.
ctggctcaggataactacaggtacat

SEQ ID NO: 38 - DNA, 19 bp, Mus sp.
gcctgggagaccctccttt

SEQ ID NO: 39 - DNA, 20 bp, Mus sp.
agcgaatggaagcccttaca

SEQ ID NO: 40 - DNA, 20 bp, Mus sp.
ctcatcgaagtggaccggca

SEQ ID NO: 41 - DNA, 25 bp, Mus sp.
ggtgaaaagaaagctaacctctttc

SEQ ID NO: 42 - DNA, 24 bp, Mus sp.
agacttgcttattcttaaatttcg

SEQ ID NO: 43 - DNA, 705 bp, Artificial Sequence
  Description of Artificial Sequence Consensus
agcttnacaccgnaggaccctgtctccaggagcacnnagctagacnncntccccngttggaggaaagctggccagctttggaatcnctgttggaagagatgnnnatgagcccangtnctttgttgttggtcttnntgctgngtctggntgtgatncctccnactctggctcagnatnannnnaggtacnnaaaattcctnnntcagcactatgatgccaagccaannggccgggannacagatactgngaaagtatgatgaagnaaagaaagctaacctcgccttgcaaagangtcaacacctttatncatgncaccaagaanaacatcanggccatctgtgganagaanggaagnccttatggagnaaacttnagaataagcaattctcnnttccagntcaccacttgnacgcacnnangagggtctcccnggcctccatgcnngtacnnngcctttaaagatttcaganatattgttattgcctgtgaagatggctggcctgtccacttcgatgagtcttttatcagtccgtagacagcaggcccctggcacagacctaggtctgttttctttttatctcccctcacagccatgatcactggttcancnttcactgtcannggccagaaaatgaattatctgaaatatacttctcctnatttataatgcacagaaataaagatatctcaaaanccanaaaaaa

SEQ ID NO: 44 - protein, 145 aa, Artificial Sequence
  Description of Artificial Sequence Consensus
MXMSPGPLXLVFXLGLVVIPPTLAQDXXRYXKFLTQHYDAKPKGRDDRYCESMMKXRKLTSPCKDVNTFIHXTKXNIKAICGXXGSPYGXNXRISXSXFQVTTCTHXGGSPRPPCXYRAXKXFRYIVIACEXGWPVHFDESFISP

SEQ ID NO: 45 - DNA, 435 bp, Mus sp.
atgacaatgagcccatgtcctttgttgttggtcttcgtgctgggtctggttgtgattcctccaactctggctcagaatgaaaggtacgaaaaattcctacgtcagcactatgatgccaagccaaagggccgggacgacagatactgtgaaagtatgatgaaggaaagaaagctaacctcgccttgcaaagatgtcaacacctttatccatggcaccaagaaaaacatcagggccatctgtggaaagaaaggaagcccttatggagaaaacttcagaataagcaattctcccttccagatcaccacttgtacgcactcaagagggtctccctggcctccatgcgggtaccgagcctttaaagatttcagatatattgttattgcctgtgaagatggctggcctgtccacttcgatgagtcttttatcagtccgtag

SEQ ID NO: 46 - DNA, 438 bp, Mus sp.
atggtgatgagcccaggttctttgttgttggtctttttgctgagtctggatgtgatccctcccactctggctcaggataactacaggtacataaaattcctgactcagcactatgatgccaagccaactggccgggattacagatactgcgaaagtatgatgaagaaaagaaagctaacctcgccttgcaaagaagtcaacacctttattcatgacaccaagaacaacatcaaggccatctgtggagagaatggaaggccttatggagtaaactttagaataagcaattctcgattccaggtcaccacttgcacgcacaaaggagggtctcccaggcctccatgccagtacaatgcctttaaagatttcagatatattgttattgcctgtgaagatggctggcctgtccacttcgatgagtcttttatcagtccgtag

SEQ ID NO: 47 - DNA, 438 bp, Mus sp.
atggcgataagcccaggcccgttgttcttgatcttcgtgctgggtctggttgtgatccctcccactctggctcaggatgactccaggtacacaaaattcctgactcagcaccatgacgccaagccaaagggccgggacgacagatactgtgaacgtatgatgaagagaagaagcctaacctcaccctgcaaagatgtcaacacctttatccatggcaacaagagcaacatcaaggccatctgtggagcgaatggaagcccttacagagaaaacttaagaatgagcaagtctcccttccaggtcaccacttgcaagcacacaggagggtctccccggcctccatgccagtaccgagcctctgcagggttcagacatgttgttattgcctgtgagaatggcttgccggtccacttcgatgagtcatttttcagtctatag

SEQ ID NO: 48 - DNA, 438 bp, Mus sp.
atggcgatgagcccaggtcctttgttcttggtcttcctgttgggtctggttgtgatccctcccactctgtctcaggatgactccaggtacacaaaattcctgactcagcactatgatgccaagccaaaaggccgggacgacagatactgcgaaagtatgatggtgaaaagaaagctaacctctttctgcaaagatgtcaacacctttatccatgacaccaagaacaacatcaaggccatctgtggaaagaaaggaagcccttatggacgaaatttaagaataagcaagtctcgcttccaggtcaccacttgcacacacaaaggaaggtctccccggcctccatgcaggtaccgagcctctaaagggttcagatatattattattggctgtgagaatggctggcctgtccactttgatgagtcttttatcagtccatag

SEQ ID NO: 49 - DNA, 438 bp, Artificial Sequence
  Description of Artificial Sequence Consensus
atggcgatgagcccaggtcctttgttnttggtcttcntgctgggtctggttgtgatccctcccactctggctcaggatgactccaggtacanaaaattcctgactcagcactatgatgccaagccaaanggccgggacgacagatactgngaaagtatgatgaagaaaagaaagctaacctcnccntgcaaagatgtcaacacctttatccatgncaccaagaacaacatcaaggccatctgtgganagaanggaagcccttatggagnaaacttnagaataagcaantctcncttccaggtcaccacttgcacgcacanaggagggtctcccnggcctccatgcnngtaccgagcctntaaagnnttcagatatattgttattgcctgtgannatggctggcctgtccacttcgatgagtcttttatcagtccntag